SEQUENCE LISTING (35 sequences)

SEQ ID NO: 1 - protein, 552 aa, Homosapien
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 2 - DNA, 8789 bp, Homosapien
atgctcagtggcttctcgacaagttggcagcaacaacacggccctggtcgtcgtcgccgctgcggtaacggagcggtttgggtggcggagcctgcgttcgcgccttcccgctctcctcgggaggcccttcctgctctcccctaggctccgcggccgcccagggggtgggagcgggtgaggggagccaggcgcccagcgagagaggccccccgccgcagggcggcccgggagctcgaggcggtccggcccgcgcgggcagcggcgcggcgctgaggaggggcggcctggccgggacgcctcggggcgggggccgaggagctctccgggccgccggggaaagctacgggcccggtgcgtccgcggaccagcagcgcgggagagcggactcccctcgccaccgcccgagcccaggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttgttctgctttatcttttcataaagggttgaaaatgtgtttgctgccttgctcctagcagacagaaactggattaaaacaattttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaagtaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtctcactgttctgcaaaggtggcaatgcttaaactaaataatgaataaactgaatattttggaaactgctaaattctatgttaaatactgtgcagaataatggaaacattacagttcataataggtagtttggatatttttgtacttgatttgatgtgactttttttggtataatgtttaaatcatgtatgttatgatattgtttaaaattcagtttttgtatcttggggcaagactgcaaacttttttatatcttttggttattctaagccctttgccatcaatgatcatatcaattggcagtgactttgtatagagaatttaagtagaaaagttgcagatgtattgactgtaccacagacacaatatgtatgctttttacctagctggtagcataaataaaactgaatctcaacatacaaagttgaattctaggtttgatttttaagattttttttttcttttgcacttttgagtccaatctcagtgatgaggtaccttctactaaatgacaggcaacagccagttctattgggcagctttgtttttttccctcacactctaccgggacttccccatggacattgtgtatcatgtgtagagttggttttttttttttttaatttttattttactatagcagaaatagacctgattatctacaagatgataaatagattgtctacaggataaatagtatgaaataaaatcaaggattatctttcagatgtgtttacttttgcctggagaacttttagctatagaaacacttgtgtgatgatagtcctccttatatcacctggaatgaacacagcttctactgccttgctcagaaggtcttttaaatagaccatcctagaaaccactgagtttgcttatttctgtgatttaaacatagatcttgatccaagctacatgacttttgtctttaaataacttatctaccacctcatttgtactcttgattacttacaaattctttcagtaaacacctaattttcttctgtaaaagtttggtgatttaagttttattggcagttttataaaaagacatcttctctagaaattgctaactttaggtccattttactgtgaatgaggaataggagtgagttttagaataacagatttttaaaaatccagatgatttgattaaaaccttaatcatacattgacataattcattgcttcttttttttgagatatggagtcttgctgtgttgcccaggcaggagtgcagtggtatgatctcagctcactgcaacctctgcctcccgggttcaactgattctcctgcctcagcctccctggtagctaggattacaggtgcccgccaccatgcctggctaacttttgtagttttagtagagacggggttttgcctgttggccaggctggtcttgaactcctgacctcaagtgatccatccaccttggcctcccaaagtgctgggattacgggcgtgagccactgtccctggcctcattgttcccttttctactttaaggaaagttttcatgtttaatcatctggggaaagtatgtgaaaaatatttgttaagaagtatctctttggagccaagccacctgtcttggtttctttctactaagagccataaagtatagaaatacttctagttgttaagtgcttatatttgtacctagatttagtcacacgcttttgagaaaacatctagtatgttatgatcagctattcctgagagcttggttgttaatctatatttctatttcttagtggtagtcatctttgatgaataagactaaagattctcacaggtttaaaattttatgtctactttaagggtaaaattatgaggttatggttctgggtgggttttctctagctaattcatatctcaaagagtctcaaaatgttgaatttcagtgcaagctgaatgagagatgagccatgtacacccaccgtaagacctcattccatgtttgtccagtgcctttcagtgcattatcaaagggaatccttcatggtgttgcctttattttccggggagtagatcgtgggatatagtctatctcatttttaatagtttaccgcccctggtatacaaagataatgacaataaatcactgccatataaccttgctttttccagaaacatggctgttttgtattgctgtaaccactaaataggttgcctataccattcctcctgtgaacagtgcagatttacaggttgcatggtctggcttaaggagagccatacttgagacatgtgagtaaactgaactcatattagctgtgctgcatttcagacttaaaatccatttttgtggggcagggtgtggtgtgtaaaggggggtgtttgtaatacaagttgaaggcaaaataaaatgtcctgtctcccagatgatatacatcttattatttttaaagtttattgctaattgtaggaaggtgagttgcaggtatctttgactatggtcatctggggaaggaaaattttacattttactattaatgctccttaagtgtctatggaggttaaagaataaaatggtaaatgtttctgtgcctggtttgatggtaactggttaatagttactcaccattttatgcagagtcacattagttcacaccctttctgagagccttttgggagaagcagttttattctctgagtggaacagagttctttttgttgataatttctagtttgctcccttcgttattgccaactttactggcattttatttaatgatagcagattgggaaaatggcaaatttaggttacggaggtaaatgagtatatgaaagcaattacctctaaagccagttaacaattattttgtaggtggggtacactcagcttaaagtaatgcatttttttttcccgtaaaggcagaatccatcttgttgcagatagctatctaaataatctcatatcctcttttgcaaagactacagagaataggctatgacaatcttgttcaagcctttccatttttttccctgataactaagtaatttctttgaacataccaagaagtatgtaaaaagtccatggccttattcatccacaaagtggcatcctaggcccagccttatccctagcagttgtcccagtgctgctaggttgcttatcttgtttatctggaatcactgtggagtgaaattttccacatcatccagaattgccttatttaagaagtaaaacgttttaatttttagcctttttttggtggagttatttaatatgtatatcagaggatatactagatggtaacatttctttctgtgcttggctatctttgtggacttcaggggcttctaaaacagacaggactgtgttgcctttactaaatggtctgagacagctatggttttgaatttttagttttttttttttaacccacttcccctcctggtctcttccctctctgataattaccattcatatgtgagtgttagtgtgcctccttttagcattttcttcttctctttctgattcttcatttctgactgcctaggcaaggaaaccagataaccaaacttactagaacgttctttaaaacacaagtacaaactctgggacaggacccaagacactttcctgtgaagtgctgaaaaagacctcattgtattggcatttgatatcagtttgatgtagcttagagtgcttcctgattcttgctgagtttcaggtagttgagatagagagaagtgagtcatattcatattttcccccttagaataatattttgaaaggtttcattgcttccacttgaatgctgctcttacaaaaactggggttacaagggttactaaattagcatcagtagccagaggcaataccgttgtctggaggacaccagcaaacaacacacaacaaagcaaaacaaaccttgggaaactaaggccatttgttttgttttggtgtcccctttgaagccctgccttctggccttactcctgtacagatatttttgacctataggtgcctttatgagaattgagggtctgacatcctgccccaaggagtagctaaagtaattgctagtgttttcagggattttaacatcagactggaatgaatgaatgaaactttttgtcctttttttttctgtttttttttttctaatgtagtaaggactaaggaaaacctttggtgaagacaatcatttctctctgttgatgtggatacttttcacaccgtttatttaaatgctttctcaataggtccagagccagtgttcttgttcaacctgaaagtaatggctctgggttgggccagacagttgcactctctagtttgccctctgccacaaatttgatgtgtgacctttgggcaagtcatttatcttctctgggccttagttgcctcatctgtaaaatgagggagttggagtagattaattattccagctctgaaattctaagtgaccttggctaccttgcagcagttttggatttcttccttatctttgttctgctgtttgagggggctttttacttatttccatgttattcaaaggagactaggcttgatattttattactgttcttttatggacaaaaggttacatagtatgcccttaagacttaattttaaccaaaggcctagcaccaccttaggggctgcaataaacacttaacgcgcgtgcgcacgcgcgcgcgcacacacacacacacacacacacacacacacaggtcagagtttaaggctttcgagtcatgacattctagcttttgaattgcgtgcacacacacacgcacgcacacactctggtcagagtttattaaggctttcgagtcatgacattatagcttttgagttggtgtgtgtgacaccaccctcctaagtggtgtgtgcttgtaattttttttttcagtgaaaatggattgaaaacctgttgttaatgcttagtgatattatgctcaaaacaaggaaattcccttgaaccgtgtcaattaaactggtttatatgactcaagaaaacaataccagtagatgattattaactttattcttggctctttttaggtccattttgattaagtgacttttggctggatcattcagagctctcttctagcctacccttggatgagtacaattaatgaaattcatattttcaaggacctgggagccttccttggggctgggttgagggtggggggttggggagtcctggtagaggccagctttgtggtagctggagaggaagggatgaaaccagctgctgttgcaaaggctgcttgtcattgatagaaggactcacgggcttggattgattaagactaaacatggagttggcaaactttcttcaagtattgagttctgttcaatgcattggacatgtgatttaagggaaaagtgtgaatgcttatagatgatgaaaacctggtgggctgcagagcccagtttagaagaagtgagttgggggttggggacagatttggtggtggtatttcccaactgtttcctcccctaaattcagaggaatgcagctatgccagaagccagagaagagccactcgtagcttctgctttggggacaactggtcagttgaaagtcccaggagttcctttgtggctttctgtatacttttgcctggttaaagtctgtggctaaaaaatagtcgaacctttcttgagaactctgtaacaaagtatgtttttgattaaaagagaaagccaactaaaaaaaaaaaaaaaaaaaa

SEQ ID NO: 3 - RNA, 19 nt, Artificial
  SMAD4 shRNA
guaaguagcuggcugacca

SEQ ID NO: 4 - DNA, 19 bp, Homosapien
tggtcagccagctacttac

SEQ ID NO: 5 - DNA, 21 bp, Artificial
  Scramble shRNA
aagttgcaaatcgcgtctcta

SEQ ID NO: 6 - RNA, 19 nt, Artificial
  SMAD4 shRNA
agaagugagucauauucau

SEQ ID NO: 7 - DNA, 19 bp, Homosapien
atgaatatgactcacttct

SEQ ID NO: 8 - protein, 142 aa, Artificial
  recombinant CLP/cotl 1
MATKIDKEACRAAYNLVRDDGSAVIWVTFKYDGSTIVPGEQGAEYQHFIQQCTDDVRLFAFVRFTTGDAMSKRSKFALITWIGENVSGLQRAKTGTDKTLVKEVVQNFAKEFVISDRKELEEDFIKSELKKAGGANYDAQTE

SEQ ID NO: 9 - DNA, 20 bp, Artificial
  primer
ctggataccgcagctaggaa

SEQ ID NO: 10 - DNA, 20 bp, Artificial
  primer
ccctcttaatcatggcctca

SEQ ID NO: 11 - DNA, 17 bp, Artificial
  primer
tgcggccgattgtgaac

SEQ ID NO: 12 - DNA, 21 bp, Artificial
  primer
cctcttttctctgcggaacgt

SEQ ID NO: 13 - DNA, 20 bp, Artificial
  primer
ctaccatggaccatcctgct

SEQ ID NO: 14 - DNA, 20 bp, Artificial
  primer
cctatcccaaggtcttgctg

SEQ ID NO: 15 - DNA, 20 bp, Artificial
  primer
gtggacgaggcaagagtttc

SEQ ID NO: 16 - DNA, 20 bp, Artificial
  primer
ttcccgaggtccatctactg

SEQ ID NO: 17 - DNA, 20 bp, Artificial
  primer
tcaggaacctcaccttggac

SEQ ID NO: 18 - DNA, 20 bp, Artificial
  primer
gcacagtggggagaagagag

SEQ ID NO: 19 - DNA, 20 bp, Artificial
  primer
gattgatgacaagggcatgg

SEQ ID NO: 20 - DNA, 20 bp, Artificial
  primer
cccatagggtgagaaaacca

SEQ ID NO: 21 - DNA, 20 bp, Artificial
  primer
atcgccactccagctacatc

SEQ ID NO: 22 - DNA, 20 bp, Artificial
  primer
ggcagcatcacagtagcatc

SEQ ID NO: 23 - DNA, 20 bp, Artificial
  primer
ccatttccaatcatcctgct

SEQ ID NO: 24 - DNA, 20 bp, Artificial
  primer
acctttgcctatgtgcaacc

SEQ ID NO: 25 - DNA, 20 bp, Artificial
  primer
ggaggacgtgaatgacaaca

SEQ ID NO: 26 - DNA, 23 bp, Artificial
  primer
cacgttctcacacgtttctttac

SEQ ID NO: 27 - DNA, 20 bp, Artificial
  primer
ggccagtgcaactctttcta

SEQ ID NO: 28 - DNA, 21 bp, Artificial
  primer
ctgtagttcagggcagttgag

SEQ ID NO: 29 - DNA, 20 bp, Artificial
  primer
gttatcctcgagggcatggt

SEQ ID NO: 30 - DNA, 20 bp, Artificial
  primer
cgtcctccagagtcagcact

SEQ ID NO: 31 - DNA, 22 bp, Artificial
  primer
aactgttagacgacggacatag

SEQ ID NO: 32 - DNA, 20 bp, Artificial
  primer
cttcggtctggtgtccattt

SEQ ID NO: 33 - DNA, 22 bp, Artificial
  primer
tgaaccatccaacgacaatcta

SEQ ID NO: 34 - DNA, 21 bp, Artificial
  primer
cttgctgaggtaccatgttct

SEQ ID NO: 35 - protein, 32 aa, Artificial
  SBD peptide
APGLPNGLLSGDEDFSSIADMDFSALLSQISS

(19) United States
(12) Patent Application Publication
(10) Pub. No.: US 2022/0025379 A1
(43) Pub. Date: Jan. 27, 2022

(54) METHODS OF TREATING SCHIZOPHRENIA AND OTHER NEUROPSYCHIATRIC DISORDERS

(71) Applicant: UNIVERSITY OF ROCHESTER, Rochester, NY (US)

(72) Inventors: Steven A. GOLDMAN, Webster, NY (US); Zhengshan LIU, Rochester, NY (US)

(21) Appl. No.: 17/413,384

(22) PCT Filed: Dec. 11, 2019
(86) PCT No.: PCT/US2019/065742

§ 371 Date: Jun. 11, 2021

Related U.S. Application Data
(60) Provisional application No. 62/778,145, filed on Dec. 11, 2018.

(51) Int. Cl. C12N 15/113; A61P 25/18; A61K 31/713

(52) U.S. Cl. CPC C12N 15/1138; A61P 25/18; C12N 2310/122; C12N 2310/14; C12N 2310/531; A61K 31/713

(57) ABSTRACT

The present disclosure relates to methods of restoring K⁺ uptake by glial cells in a subject. These methods involve administering, to the subject, a SMAD4 inhibitor under conditions effective to restore K⁺ uptake by said glial cells. The present disclosure is also directed to methods of treating or inhibiting the onset of a neuropsychiatric disorder in a subject. These methods involve administering, to a subject in need thereof, a SMAD4 inhibitor under conditions effective to treat or inhibit the onset of the neuropsychiatric disorder in the subject.

This application claims the benefit of U.S. Provisional Patent Application Ser. No. 62/778,145, filed Dec. 11, 2018, which is hereby incorporated by reference in its entirety.

This invention was made with government support under MH099578 awarded by National Institutes of Health. The government has certain rights in the invention.

### FIELD OF THE DISCLOSURE

The present disclosure relates to methods for restoring glial cell potassium (K⁺) uptake in glial cells having impaired K⁺ channel function. These methods are suitable for treating a subject suffering from a neuropsychiatric condition.

### BACKGROUND

Schizophrenia is a psychiatric disorder characterized by delusional thought, auditory hallucination and cognitive impairment, which affects roughly 1% of the population worldwide, and yet remains poorly understood (Allen et al., “Systematic Meta-Analyses and Field Synopsis of Genetic Association Studies in Schizophrenia: The SzGene Database,” Nature Genetics 40:827-834 (2008); Sawa & Snyder, “Schizophrenia: Diverse Approaches to a Complex Disease,” Science 296:692-695 (2002)). Over the past decade, it has become clear that a number of schizophrenia-associated genes are involved in the development and physiology of glial cells (Yin et al., “Synaptic Dysfunction in Schizophrenia,” Adv. Exp. Med. Biol. 970:493-516 (2012)). Accordingly, both astrocytic and oligodendrocytic dysfunction has been implicated in the etiology of schizophrenia. Astrocytes in particular have essential roles in both the structural development of neural networks as well as the coordination of neural circuit activity, the latter through their release of glial transmitters, maintenance of synaptic density, and regulation of synaptic potassium and neurotransmitter levels (Christopherson et al., “Thrombospondins are Astrocyte-Secreted Proteins That Promote CNS Synaptogenesis,” Cell 120: 421-433 (2005); Chung et al., “Astrocytes Mediate Synapse Elimination Through MEGF10 and MERTK Pathways,” Nature 504:394-400 (2013); and Thrane et al., “Ammonia Triggers Neuronal Disinhibition and Seizures by Impairing Astrocyte Potassium Buffering,” Nat. Med. 19:1643-1648 (2013)). However, the role that astrocyte dysfunction plays in the development of neuropsychiatric disorders, such as schizophrenia, is unknown. The present disclosure is aimed at overcoming this and other deficiencies in the art.

### SUMMARY

A first aspect of the present disclosure relates to a method of restoring K⁺ uptake by glial cells, where said glial cells have impaired K⁺ channel function. This method involves administering, to the glial cells having impaired K⁺ channel function, a SMAD4 inhibitor under conditions effective to restore K⁺ uptake by said glial cells.

Another aspect of the present disclosure relates to a method of restoring K⁺ uptake by glial cells in a subject. This method involves selecting a subject having impaired glial cell K⁺ uptake, and administering, to the selected subject, a SMAD4 inhibitor under conditions effective to restore K⁺ uptake by said glial cells.

Another aspect of the present disclosure relates to a method of treating or inhibiting the onset of a neuropsychiatric disorder in a subject. This method involves selecting a subject having or at risk of having a neuropsychiatric disorder, and administering, to the selected subject, a SMAD4 inhibitor under conditions effective to treat or inhibit the onset of the neuropsychiatric disorder in the subject.

To investigate the role of glial pathology in neurological and neuropsychiatric disorders like schizophrenia, a protocol for generating glial progenitor cells (GPCs) from induced pluripotent cells (iPSCs) was established (Wang et al., “Human iPSC-Derived Oligodendrocyte Progenitor Cells Can Myelinate and Rescue a Mouse Model of Congenital Hypomyelination,” Cell Stem Cell 12:252-264 (2013), which is hereby incorporated by reference in its entirety). This model permits the generation of GPCs and their derived astrocytes and oligodendrocytes from patients with schizophrenia, in a manner that preserves their genetic integrity and functional repertoires. This protocol has provided a means by which to assess the differentiation, gene expression and physiological function of astrocytes derived from patients with schizophrenia, both in vitro, and in vivo after engraftment into immune deficient mice (Windrem et al., “Human iPSC Glial Mouse Chimeras Reveal Glial Contributions to Schizophrenia,” Cell Stem Cell 21:195-208.e6 (2017), which is hereby incorporated by reference in its entirety). It was noted that such human glial chimeric mice, colonized with iPSC-derived GPCs generated from schizophrenic patients, exhibited profound abnormalities in both astrocytic differentiation and mature structure that were associated with significant physiological and behavioral abnormalities. Importantly, RNA sequence analysis revealed that the developmental defects in these schizophrenia GPCs were associated with the down-regulation of a core set of differentiation-associated genes, whose transcriptional targets included a host of transporters, channels and synaptic modulators found similarly deficient in schizophrenia glia.

As described herein, targetable signaling nodes at which such schizophrenia-associated glial pathology might be moderated were identified. To that end, iPSC GPCs were generated from patients with childhood-onset schizophrenia or from their normal controls (CTR), and astrocytes were produced from these. Both patterns of gene expression and astrocytic functional differentiation by schizophrenic- and control-derived GPCs were compared. It was found that excessive TGFβ signaling plays a critical role in the dysregulated differentiation of schizophrenia-derived GPCs, that TGFβ's actions in this cellular context were signaled through SMAD4, and that aspects of phenotypic normalcy could be restored to SCZ glia by SMAD4 inhibition.

### BRIEF DESCRIPTION OF THE DRAWINGS

FIGS. 1A-1F show efficient generation of hGPCs from SCZ iPSCs. Flow cytometry revealed that >90% of undifferentiated hiPSCs expressed SSEA4 in both SCZ (4 SCZ lines, n≥3/each line)- and CTR (4 CTR lines, n≥3/each line)-derived hiPSCs (FIG. 1A). At the neural progenitor cell (NPC) stage, the expression of the NPC marker CD133 was no different between SCZ- and CTR-derived lines (FIG. 1B). CD140a-defined hGPCs were likewise similarly generated from both SCZ- and CTR-derived iPSCs, and the relative proportion of CD140a⁺ cells was no different in SCZ and CTR hGPC cultures (FIG. 1C). At the astrocytic progenitor stage, the expression of CD44 was no different between SCZ- and CTR-derived lines (FIG. 1D). After being cultured with BMP4, the percentage of PDGFaR⁺ glia was significantly higher in SCZ lines (4 SCZ lines, n≥3/each line) compared to CTR lines (4 CTR lines, n≥3/each line) (FIG. 1E). In addition to GFAP, the proportion of S10013⁺ astrocytes was also significantly higher in CTR lines relative to SCZ lines (FIG. 1F). FSC, forward scatter. Scale: 50 μm. ***p<0.001 by two tailed t-test; NS: not significant; means±SEM.

FIGS. 2A-2J show that astrocytic differentiation was impaired in SCZ GPCs. As shown in FIGS. 2A-2D, at the neural progenitor cell (NPC) stage, both SCZ and CTR (4 distinct patients and derived lines each, n≥3/each line) hNPCs highly expressed both SOX1 and PAX6. Similarly, the efficiency of PDGFRα/CD140a-defined hGPC generation did not differ between SCZ and CTR lines (4 different patient-specific lines each, n≥3/each line) (FIGS. 2E-2G). In contrast, as shown in FIGS. 2H-2J, the proportion of GFAP⁺ astrocytes was significantly higher in CTR lines (4 CTR lines, n≥3/each line [70.1±2.4%]) vs. SCZ lines (4 SCZ lines, n≥3/each line, [39.9±2.0%]). Scale: 50 μm; ***p<0.001 by two tailed t-test; NS: not significant; mean±SEM.

FIGS. 3A-3E show TGFβ signal-dependent transcripts were upregulated in SCZ GPCs. FIG. 3A is a schematic of the Ingenuity Pathway Analysis of RNA-seq data, which revealed that TGFβ-dependent transcription was upregulated in SCZ hGPCs. Upregulated genes included LTBP1, LTBP2, IGFBP3, TGFB1, PDGFB, GDF3, GDF7, BMP1 and BMP5. Down regulated genes included AMH, and BMP3. qPCR confirmed that TGFβ pathway-associated and regulated genes, including BMP1, BMPR2, RUNX2, SERPINE1, BAMBI, among others, were significantly upregulated in SCZ hGPCs (4 SCZ lines, 3 repeats/each line), relative to CTR cells (4 CTR lines, 3 repeats/each line) (FIG. 3B). In contrast, as shown in FIG. 3C, the expression of these genes did not differ between SCZ and CTR lines at NPC stage. Principal component analysis (PCA) showed similar methylation state between CTR- and SCZ-derived iPSCs (FIG. 3D). FIG. 3E is a heatmap, which indicates that the variability in iPSC methylation state was primarily due to sex and individual line (p<0.05), rather than to either disease state or age. *p<0.05, **p<0.01 by two tailed t-tests; NS: not significant; mean±SEM.

FIGS. 4A-4B show the validation of BAMBI overexpression and knockdown. In CTR hGPCs (4 CTR lines, 3 repeats/each line) transduced with lentiviral-BAMBI, qPCR confirmed that BAMBI was significantly overexpressed (FIG. 4A). Whereas SCZ hGPCs (4 SCZ lines, 3 repeats/each line) expressed high levels of BAMBI relative to CTR hGPCs, lentiviral-BAMBI-shRNAi transduction of SCZ hGPCs suppressed BAMBI expression to the level of CTR hGPCs (FIG. 4B). ***P<0.001 by one-way ANOVA for A and B; mean±SEM.

FIGS. 5A-5C show that BAMBI expression in normal hGPCs phenocopied the glial differentiation defect of SCZ. FIGS. 5A-5B show that overexpression of the membrane-bound BMP antagonist BAMBI in CTR hGPCs (4 CTR lines, 3 repeats/each line) significantly decreased their efficiency of astrocytic transition. However, BAMBI knockdown in SCZ hGPCs (4 SCZ lines, 3 repeats/each line) was not sufficient to restore astrocytic differentiation (FIG. 5B). Besides BAMBI, the BMP antagonists follistatin (FST) and gremlin1 (GREM1) were also upregulated in SCZ hGPCs, relative to controls (FIG. 5C). Scale: 50 μm; ***p<0.001, 1-way ANOVA for B; **P<0.001 by 2-tailed t-test for C; NS: not significant; mean±SEM.

FIGS. 6A-6D show SMAD4 regulated the astrocytic differentiation of SCZ GPCs. FIG. 6A is a schematic depiction of SMAD4 regulating the expression of TGFβ and BMP pathway signaling through: 1) phosphorylation of both SMAD2/3 and SMAD1/5/8; 2) SMAD nuclear translocation and activation of target promoters, including the early induction of the endogenous BMP inhibitors BAMBI, follistatin (FST) and gremlin1 (GREM1); and 3) their subsequent feedback inhibition of BMP signals. The graphs of FIG. 6B show that BAMBI, FST and GREM1 were all significantly over-expressed in SCZ CD140a-sorted hGPCs relative to control-derived hGPCs. SMAD4 knockdown in SCZ hGPCs (4 SCZ lines, 3 repeats/line) then repressed the expression of BAMBI, FST and GREM1 to control levels. FIG. 6C is a panel of immunocytochemical images showing SMAD4 knockdown in SCZ hGPCs restored astrocytic differentiation to that of CTR hGPCs (4 SCZ lines, 3 repeats/each line). DOX (−)/(+) means short-term/long-term culture with DOX. SMAD4 knock-down after astrocytic induction, as mediated via continuous doxycycline exposure, caused a loss of GFAP-defined astrocytes in both SCZ and CTR groups as shown in the graph of FIG. 6D. DOX (−)/(+) means short-term/long-term culture with DOX. Scale: 50 μm; *p<0.05, **p<0.01, ***p<0.001; one-way ANOVA; NS: not significant; mean±SEM.

FIGS. 7A-7C shows validation of SMAD4 knockdown. FIG. 7A are graphs showing that SMAD4 mRNA levels were no different between SCZ and control hGPCs and astrocytes, as reflected in CD140a-sorted hGPCs (left plot) and CD44-sorted astrocytes (right). FIG. 7B is a schematic of the experimental plan, for assessing the effects of transient, doxycycline-regulated SMAD4 knock-down on astrocytic differentiation by SCZ and CTR patient-derived hGPCs. FIG. 7C is a graph showing SMAD4 expression. SCZ CD140a-sorted hGPCs (4 SCZ lines, 3 repeats/each line) were transduced with doxycycline (DOX)-inducible lentivirus-SMAD4-shRNAi, which was then induced by DOX to drive the expression of SMAD4-shRNAi. The cultures were then switched to astrocyte differentiation conditions, and DOX either withdrawn, allowing SMAD4 expression with astrocytic maturation (DOX only in GPC stage), or sustained, thereby continuing to inhibit SMAD4 expression during astrocytic maturation (DOX maintained in AST stage). The lentiviral SMAD4-shRNAi strongly repressed SMAD4 expression under DOX, whereas SMAD4 expression was unaffected in the absence of DOX induction. DOX (−)/(+) means short-term/long-term culture with DOX. **P<0.01 by one-way ANOVA; NS: not significant; mean±SEM.

FIGS. 8A-8B show potassium channel (KCN)-associated gene expression in SCZ hGPCs. FIG. 8A is a heat map showing the differentially expressed potassium channel genes in SCZ-derived hGPC lines. Each SCZ-derived hGPC line was individually compared against three pooled CTR-derived hGPC lines (FDR 5%, FC>2.00 [if applicable]). Genes shown were found differentially expressed in at least three out of four assessed SCZ-derived hGPC lines. qPCR confirmed that potassium channel-associated genes including ATP1A2, SLC12A6, and KCNJ9 were all significantly downregulated in SCZ hGPCs (4 SCZ lines, 3 repeats/each line), relative to CTR cells (4 CTR lines, 3 repeats/each (FIG. 8B) line). **p<0.01 by two tailed t-test; mean±SEM.

FIGS. 9A-9E shows potassium uptake was decreased by SCZ astrocytes. FIG. 9A is a schematic depiction of the Na+/K+-ATPase pump, NKCC1 Na⁺/K⁺/2Cl⁻ cotransporter, and inwardly rectifying K+ channels involvement in the regulation of potassium uptake by astrocytes. qPCR confirmed that several K+ channel-associated genes were down-regulated in SCZ CD44+ astrocyte-biased GPCs relative to CTR cells as shown in the graphs of FIG. 9B. SCZ and CTR CD44+ GPCs were cultured in FBS with BMP4 to produce mature GFAP+ astrocytes, which were then assessed for K⁺ uptake; the results were normalized to both total protein and cell number. FIG. 9C shows K⁺ uptake by SCZ astrocytes was significantly reduced (4 SCZ lines, 5 repeats/each line), compared to K⁺ uptake by CTR astrocytes (4 CTR lines, 5 repeats/each line). Astrocytes were treated with ouabain, bumetanide, and tertiapin to assess which potassium transporter classes were functionally impaired in SCZ astrocytes relative to control (4 lines of each, 4 repeats/line). Both ouabain and bumetanide efficiently decreased K⁺ uptake by CTR astrocytes (FIG. 9D, gray bars), whereas neither affected K+ uptake by SCZ astrocytes (FIG. 9E, purple bars). *P<0.05, **P<0.01, ***P<0.001 by two tailed t-test for B and C; ***P<0.001 by one-way ANOVA for D; NS: not significant; mean±SEM.

FIGS. 10A-10C show generation of astrocytes from SCZ CD44+ astrocyte-biased progenitors. Both SCZ-derived and CTR-derived CD44+ astrocytic precursors were induced to differentiate into astrocytes. Immunostaining for GFAP demonstrated that the efficiencies of astrocytic generation were not significantly different between SCZ-derived lines (FIG. 10A, right image; 4 SCZ lines, 5 repeats/each line) and CTR-derived lines (FIG. 10A, left image; 4 CTR lines, 5 repeats/each line) (see also graph of FIG. 10B). qPCR revealed no difference in GFAP mRNA expression between SCZ- and CTR-derived CD44+ astrocytic precursors as depicted in FIG. 10C. Scale: 50 μm. Two tailed t-tests for B and C; NS: not significant; mean±SEM.

### DETAILED DESCRIPTION

A first aspect of the present disclosure relates to a method of restoring K+ uptake by glial cells, where said glial cells have impaired K⁺ channel function. This method involves administering, to the glial cells having impaired K⁺ channel function, a SMAD4 inhibitor under conditions effective to restore K⁺ uptake by said glial cells.

Another aspect of the present disclosure relates to a method of restoring K⁺ uptake by glial cells in a subject. This method involves selecting a subject having impaired glial cell K⁺ uptake, and administering, to the selected subject, a SMAD4 inhibitor under conditions effective to restore K⁺ uptake by said glial cells.

As referred to herein, “glial cells” encompass glial progenitor cells, oligodendrocyte-biased progenitor cells, astrocyte-biased progenitor cells, oligodendrocytes, and astrocytes. Glial progenitor cells are bipotential progenitor cells of the brain that are capable of differentiating into both oligodendrocytes and astrocytes. Glial progenitor cells can be identified by their expression of certain stage-specific surface antigens, such as the ganglioside recognized by the A2B5 antibody and PDGFRα (CD140a), as well as stage-specific transcription factors, such as OLIG2, NKX2.2, and SOX10. Oligodendrocyte-biased and astrocyte-biased progenitor cells are identified by their acquired expression of stage selective surface antigens, including, for example CD9 and the lipid sulfatide recognized by the 04 antibody for oligodendrocyte-biased progenitor cells, and CD44 for astrocyte-biased progenitors. Mature oligodendrocytes are identified by their expression of myelin basic protein, and mature astrocytes are most commonly identified by their expression of glial fibrillary acidic protein (GFAP). In one embodiment of the methods described herein, K+ uptake is restored in glial progenitor cells. In another embodiment, K⁺ uptake is restored in astrocyte-biased progenitor cells. In another embodiment, K⁺ uptake is restored in astrocytes.

In accordance with these aspects of the present disclosure, glial cells having impaired K⁺ uptake, are glial cells, in particular, glial progenitor cells, astrocyte-biased progenitor cells, and/or astrocytes, having reduced K⁺ uptake as compared to normal, healthy glial cells. In one embodiment, glial cells having reduced K⁺ uptake are glial cells where one or more potassium channel encoding genes is down regulated, causing a reduction in the corresponding potassium channel protein expression. In particular, a down regulation in expression of one or more potassium channel encoding genes selected from KCNJ9, KCNH8, KCNA3, KCNK9, KCNC1, KCNC3, KCNB1, KCNF1, KCNA6, SCN3A, SCN2A, SCNN1D, SCN8A, SCN3B, SLC12A6, SLC6A1, SLC8A3, ATP1A2, ATP1A3, ATP2B2 can lead to a reduction in glial cell K⁺ uptake.

Thus, in one embodiment, selecting a subject having impaired glial cell K⁺ uptake involves assessing potassium uptake by glial cells of the subject, comparing the level of potassium uptake by said glial cells to the level of potassium uptake by a population of control, healthy glial cells, and selecting the subject having a reduction in glial cell K⁺ uptake. In another embodiment, selecting a subject having impaired glial cell K⁺ uptake involves assessing glial cell expression level of one or more potassium channel encoding genes selected from the group consisting of KCNJ9, KCNH8, KCNA3, KCNK9, KCNC1, KCNC3, KCNB1, KCNF1, KCNA6, SCN3A, SCN2A, SCNN1D, SCN8A, SCN3B, SLC12A6, SLC6A1, SLC8A3, ATP1A2, ATP1A3, ATP2B2, and selecting the subject if there is a downregulation in the expression of the one or more potassium channel encoding genes. In another embodiment, selecting a subject having impaired glial cell K⁺ uptake involves assessing glial cell protein expression of one or more potassium channels including, GIRK-3 (encoded by KCNJ9), potassium voltage-gated channel subfamily H member 8 (encoded by KCNH8), potassium voltage-gated channel subfamily A member 3 (encoded by KCNA3), potassium channel subfamily K member 9 (encoded by KCNK9), potassium voltage-gated channel subfamily C member 1 (encoded by KCNC1), potassium voltage-gated channel subfamily C member 3 (encoded by KCNC3), potassium voltage-gated channel subfamily B member 1 (encoded by KCNB1), potassium voltage-gated channel subfamily F member 1 (encoded by KCNF1), potassium voltage-gated channel subfamily A member 6 (encoded by KCNA6), Sodium channel protein type 3 subunit alpha (encoded by SCN3A), sodium channel protein type 2 subunit alpha (encoded by SCN2A), amiloride-sensitive sodium channel subunit delta (encoded by SCNN1D), sodium channel protein type 8 subunit alpha (encoded by SCN8A), sodium channel subunit beta-3 (encoded by SCN3B), solute carrier family 12 member 6 (i.e., K⁺/Cl⁻ cotransporter 3) (encoded by SLC12A6), sodium- and chloride-dependent GABA transporter 1 (i.e., GAT-1) (encoded by SLC6A Na⁺/Ca⁺² exchanger 3 (encoded by SLC8A3), Na⁺/K⁺-transporting ATPase subunit alpha-2 (encoded by ATP1A2), Na⁺/K⁺- transporting ATPase subunit alpha-2 (encoded by ATP1A3), plasma membrane calcium-transporting ATPase 2 (i.e., PMCA2) (encoded by ATP2B2). The subject is selected for treatment using the methods as described herein if there is a decrease in the level of one or more potassium channel proteins.

Potassium uptake, potassium channel gene expression, potassium channel protein expression, and SMAD4 gene expression can each be assessed using methods described herein and that are well known to those of skill in the art. These parameters can be assessed in a glial cell sample taken from a subject. Alternatively, one or more of these parameters can be assessed in a glial cell sample derived from induced pluripotent stem cells (iPSCs) derived from the subject. iPSCs can be obtained from virtually any somatic cell of the subject, including, for example, and without limitation, fibroblasts, such as dermal fibroblasts obtained by a skin sample or biopsy, synoviocytes from synovial tissue, keratinocytes, mature B cells, mature T cells, pancreatic β cells, melanocytes, hepatocytes, foreskin cells, cheek cells, lung fibroblasts, peripheral blood cells, bone marrow cells, etc. iPSCs may be derived by methods known in the art including the use of integrating viral vectors (e.g., lentiviral vectors, inducible lentiviral vectors, and retroviral vectors), excisable vectors (e.g., transposon and foxed lentiviral vectors), and non-integrating vectors (e.g., adenoviral and plasmid vectors) to deliver the aforementioned genes that promote cell reprogramming (see e.g., Takahashi and Yamanaka, Cell 126:663-676 (2006); Okita. et al., Nature 448:313-317 (2007); Nakagawa et al., Nat. Biotechnol. 26:101-106 (2007); Takahashi et al., Cell 131:1-12 (2007); Meissner et al. Nat. Biotech. 25:1177-1181 (2007); Yu et al. Science 318:1917-1920 (2007); Park et al. Nature 451:141-146 (2008); and U.S. Patent Application Publication No. 2008/0233610, which are hereby incorporated by reference in their entirety). Other methods for generating IPS cells include those disclosed in WO2007/069666, WO2009/006930, WO2009/006997, WO2009/007852, WO2008/118820, U.S. Patent Application Publication Nos. 2011/0200568 to Ikeda et al., 2010/0156778 to Egusa et al., 2012/0276070 to Musick, and 2012/0276636 to Nakagawa, Shi et al., Cell Stem Cell 3(5): 568-574 (2008), Kim et al., Nature 454: 646-650 (2008), Kim et al., Cell 136(3):411-419 (2009), Huangfu et al., Nature Biotechnology 26: 1269-1275 (2008), Zhao et al., Cell Stem Cell 3: 475-479 (2008), Feng et al., Nature Cell Biology 11: 197-203 (2009), and Hanna et al., Cell 133(2): 250-264 (2008), which are hereby incorporated by reference in their entirety. Methods of driving the iPSCs toward a glial progenitor cell (GPC) fate and on to an astrocyte fate are described herein and known in the art, see e.g., Wang et al., “Human iPSC-Derived Oligodendrocyte Progenitor Cells can Myelinate and Rescue a Mouse Model of Congenital Hypomyelination,” Cell Stem Cell 12:252-264 (2013), which is hereby incorporated by reference in its entirety.

In one embodiment, glial cells having impaired K⁺ uptake are glial cells of a subject having a neuropsychiatric disorder. A “neuropsychiatric disorder” as referred to herein, includes any brain disorder with psychiatric symptoms including, but not limited to, dementia, amnesic syndrome, and personality-behavioral changes. Neuropsychiatric disorders known to involve impaired K⁺ channel function in glial cells that are suitable for treatment using the methods described herein include, without limitation, schizophrenia, autism spectrum disorders, and bipolar disorder.

Thus, another aspect of the present disclosure relates to a method of treating or inhibiting the onset of a neuropsychiatric disorder in a subject. This method involves selecting a subject having or at risk of having a neuropsychiatric disorder, and administering, to the selected subject, a SMAD4 inhibitor under conditions effective to treat or inhibit the onset of the neuropsychiatric disorder in the subject.

In one embodiment, the subject treated in accordance with this disclosure is a subject having or at risk of having schizophrenia. Schizophrenia is a chronic and severe mental disorder that affects how an individual thinks, feels, and behaves. To date, there have been several suggested staging models of the disorder (Agius et al., “The Staging Model in Schizophrenia, and its Clinical Implications,” Psychiatr. Danub. 22(2):211-220 (2010); McGorry et al., “Clinical Staging: a Heuristic Model and Practical Strategy for New Research and Better Health and Social Outcomes for Psychotic and Related Disorders,” Can. J. Psychiatry 55(8):486-497 (2010); Fava and Kellner, “Staging: a Neglected Dimension in Psychiatric Classification,” Acta Psychiatr. Scand. 87:225-230 (1993), which are hereby incorporated by reference in their entirety). However, generally, schizophrenia develops in at least three stages: the prodromal phase, the first episode, and the chronic phase. There is also heterogeneity of individuals at all stages of the disorder, with some individuals considered ultra-high risk, clinical-high risk, or at-risk for the onset of psychosis (Fusar-Poli et al., “The Psychosis High-Risk State: a Comprehensive State-of-the-Art Review,” JAMA Psychiatry 70:107-120 (2013), which is hereby incorporated by reference in its entirety).

The methods described herein are suitable for treating a subject in any stage of schizophrenia, and at any risk level of psychosis, as all stages will involve impaired glial cell K⁺ uptake. For example, in one embodiment, a subject treated in accordance with the methods described herein is a subject that is at risk for developing schizophrenia. Such a subject may have one or more genetic mutations in one or more genes selected from ABCA13, ATK1, C4A, COMT, DGCR2, DGCR8, DRD2, MIR137, NOS1AP, NRXN1, OLIG2, RTN4R, SYN2, TOP3B YWHAE, ZDHHC8, or chromosome 22 (22q11) that have been associated with the development of schizophrenia and may or may not be exhibiting any symptoms of the disease. In another embodiment, the subject may be in the prodromal phase of the disease and exhibiting one or more early symptoms of schizophrenia, such as anxiety, depression, sleep disorders, and/or brief intermittent psychotic syndrome. In another embodiment, the subject being treated in accordance with the methods described herein is experiencing psychotic symptoms, e.g., hallucinations, paranoid delusions, of schizophrenia.

In another embodiment, the methods describe herein are utilized to treat a subject having autism or a related disorder. Related disorders include, without limitation, Asperger's disorder, Pervasive Developmental Disorder-Not Otherwise Specified, Childhood Disintegrative Disorder, and Rett's Disorder, which vary in the severity of symptoms including difficulties in social interaction, communication, and unusual behaviors (McPartland et al., “Autism and Related Disorders,” Handb Clin Neurol 106:407-418 (2012), which is hereby incorporated by reference in its entirety). The methods described herein are suitable for the treatment of each one of these conditions and at any stage of the condition. In one embodiment, the subject being treated in accordance with the methods described herein does not exhibit any symptoms of autism or a related condition. In another embodiment, the subject being treated exhibits one or more early symptoms of autism or a related condition. In yet another embodiment, the subject being treated in accordance with the methods described herein exhibits a multitude of symptoms of autism or a related condition.

In another embodiment, the methods describe herein are utilized to treat a subject having bipolar disorder. Bipolar disorder is a group of conditions characterized by chronic instability of mood, circadian rhythm disturbances, and fluctuations in energy level, emotion, sleep, and views of self and others. Bipolar disorders include, without limitation, bipolar disorder type I, bipolar disorder type II, cyclothymic disorder, and bipolar disorder not otherwise specified.

Generally, bipolar disorders are progressive conditions which develop in at least three stages: the prodromal phase, the symptomatic phase, and the residual phase (Kapczinski et al., “Clinical Implications of a Staging Model for Bipolar Disorders,” Expert Rev Neurother 9:957-966 (2009), and McNamara et al., “Preventative Strategies for Early-Onset Bipolar Disorder: Towards a Clinical Staging Model,” CNS Drugs 24:983-996 (2010); which are hereby incorporated by reference in their entirety). The methods described herein are suitable for treating subjects having any of the aforementioned bipolar disorders and subjects in any stage of a particular bipolar disorder. For example, in one embodiment, the subject treated in accordance with the methods described herein is a subject at the early prodromal phase exhibiting symptoms of mood lability/swings, depression, racing thoughts, anger, irritability, physical agitation, and anxiety. In another embodiment, the subject treated in accordance with the methods described herein is a subject at the symptomatic phase or the residual phase.

As used herein, the term “subject” and “patient” expressly includes human and non-human mammalian subjects. The term “non-human mammal” as used herein extends to, but is not restricted to, household pets and domesticated animals. Non-limiting examples of such animals include primates, cattle, sheep, ferrets, mice, rats, swine, camels, horses, rabbits, goats, dogs and cats.

In accordance with the present disclosure, an inhibitor of SMAD4 is administered to glial cells having impaired K⁺ channel function. In another embodiment, a SMAD4 inhibitor is administered to a subject having impaired glial cell K⁺ uptake. In another embodiment, a SMAD4 inhibitor is administered to a subject having or at risk of having a neuropsychiatric disorder that may or may not involve impaired glial cell K⁺ uptake. Smad4 (also known as Mothers Against Decapentaplegic Homolog 4 (MADH4) and DPC4) represents the most unique member of the Smad family. This protein acts as a shared hetero-oligomerization partner in complexes with the pathway-restricted Smads (Lagna et al., “Partnership between DPC4 and SMAD Proteins in TGF-beta Signalling Pathways,” Nature 383:832-836 (1996); Zhang et al., “The Tumor Suppressor Smad4/DPC 4 as a Central Mediator of Smad Function,” Curr. Biol. 7:270-276 (1997), which are hereby incorporated by reference in their entirety). It has been demonstrated that although Smad4 does not interact with the TGF-β receptor, it does perform two distinct functions within the Smad signaling cascade. Through its N-terminus Smad4 promotes the binding of the Smad complex to DNA, and through its C-terminus it provides an activation signal required for the Smad complex to stimulate transcription (Liu et al., “Dual Role of the Smad4/DPC4 Tumor Suppressor in TGFbeta-inducible Transcriptional Complexes,” Genes Dev. 11: 3157-3167 (1997), which is hereby incorporated by reference in its entirety.

SMAD4 amino acid sequence is provided
as SEQ ID NO: 1 below.

MDNMSITNTPTSNDACLSIVHSLMCHRQGGESETFAKRAIESLVKKL
KEKKDELDSLITAITTNGAHPSKCVTIQRTLDGRLQVAGRKGFPHVI
YARLWRWPDLHKNELKHVKYCQYAFDLKCDSVCVNPYHYERVVSPGI
DLSGLTLQSNAPSSMMVKDEYVHDFEGQPSLSTEGHSIQTIQHPPSN
RASTETYSTPALLAPSESNATSTANFPNIPVASTSQPASILGGSHSE
GLLQIASGPQPGQQQNGFTGQPATYHHNSTTTWTGSRTAPYTPNLPH
HQNGHLQHHPPMPPHPGHYWPVHNELAFQPPISNHPAPEYWCSIAYF
EMDVQVGETFKVPSSCPIVTVDGYVDPSGGDRFCLGQLSNVHRTEAI
ERARLHIGKGVQLECKGEGDVWVRCLSDHAVFVQSYYLDREAGRAPG
DAVHKIYPSAYIKVFDLRQCHRQMQQQAATAQAAAAAQAAAVAGNIP
GPGSVGGIAPAISLSAAAGIGVDDLRRLCILRMSFVKGWGPDYPRQS
IKETPCWIEIHLHRALQLLDEVLHTMPIADPQPLD

The nucleic acid sequence encoding SMAD4
is provided as SEQ ID NO: 2

| | |
|---|---|
| 1 | atgctcagtg gcttctcgac aagttggcag caacaacacg gccctggtcg tcgtcgccgc |
| 61 | tgcggtaacg gagcggtttg ggtggcggag cctgcgttcg cgccttcccg ctctcctcgg |
| 121 | gaggcccttc ctgctctccc ctaggctccg cggccgccca gggggtggga gcgggtgagg |
| 181 | ggagccaggc gcccagcgag agaggccccc cgccgcaggg cggcccggga gctcgaggcg |
| 241 | gtccggcccg cgcgggcagc ggcgcggcgc tgaggagggg cggcctggcc gggacgcctc |
| 301 | ggggcggggg ccgaggagct ctccgggccg ccggggaaag ctacgggccc ggtgcgtccg |
| 361 | cggaccagca gcgcgggaga gcggactccc ctcgccaccg cccgagccca ggttatcctg |
| 421 | aatacatgtc taacaatttt ccttgcaacg ttagctgttg tttttcactg tttccaaagg |
| 481 | atcaaaattg cttcagaaat tggagacata tttgatttaa aaggaaaaac ttgaacaaat |
| 541 | ggacaatatg tctattacga atacaccaac aagtaatgat gcctgtctga gcattgtgca |
| 601 | tagtttgatg tgccatagac aaggtggaga gagtgaaaca tttgcaaaaa gagcaattga |
| 661 | aagtttggta aagaagctga aggagaaaaa agatgaattg gattctttaa taacagctat |
| 721 | aactacaaat ggagctcatc ctagtaaatg tgttaccata cagagaacat tggatgggag |
| 781 | gcttcaggtg gctggtcgga aaggatttcc tcatgtgatc tatgcccgtc tctggaggtg |
| 841 | gcctgatctt cacaaaaatg aactaaaaca tgttaaatat tgtcagtatg cgtttgactt |
| 901 | aaaatgtgat agtgtctgtg tgaatccata tcactacgaa cgagttgtat cacctggaat |
| 961 | tgatctctca ggattaacac tgcagagtaa tgctccatca agtatgatgg tgaaggatga |
| 1021 | atatgtgcat gactttgagg gacagccatc gttgtccact gaaggacatt caattcaaac |
| 1081 | catccagcat ccaccaagta atcgtgcatc gacagagaca tacagcaccc cagctctgtt |
| 1141 | agccccatct gagtctaatg ctaccagcac tgccaacttt cccaacattc ctgtggcttc |
| 1201 | cacaagtcag cctgccagta tactgggggg cagccatagt gaaggactgt tgcagatagc |
| 1261 | atcagggcct cagccaggac agcagcagaa tggatttact ggtcagccag ctacttacca |
| 1321 | tcataacagc actaccacct ggactggaag taggactgca ccatacacac ctaatttgcc |
| 1381 | tcaccaccaa aacggccatc ttcagcacca cccgcctatg ccgccccatc ccggacatta |
| 1441 | ctggcctgtt cacaatgagc ttgcattcca gcctcccatt tccaatcatc ctgctcctga |
| 1501 | gtattggtgt tccattgctt actttgaaat ggatgttcag gtaggagaga catttaaggt |
| 1561 | tccttcaagc tgccctattg ttactgttga tggatacgtg gacccttctg gaggagatcg |
| 1621 | cttttgtttg ggtcaactct ccaatgtcca caggacagaa gccattgaga gagcaaggtt |
| 1681 | gcacataggc aaaggtgtgc agttggaatg taaaggtgaa ggtgatgttt gggtcaggtg |
| 1741 | ccttagtgac cacgcggtct ttgtacagag ttactactta gacagagaag ctgggcgtgc |
| 1801 | acctggagat gctgttcata agatctaccc aagtgcatat ataaaggtct ttgatttgcg |
| 1861 | tcagtgtcat cgacagatgc agcagcaggc ggctactgca caagctgcag cagctgccca |
| 1921 | ggcagcagcc gtggcaggaa acatccctgg cccaggatca gtaggtggaa tagctccagc |
| 1981 | tatcagtctg tcagctgctg ctggaattgg tgttgatgac cttcgtcgct tatgcatact |
| 2041 | caggatgagt tttgtgaaag gctggggacc ggattaccca agacagagca tcaaagaaac |
| 2101 | accttgctgg attgaaattc acttacaccg ggccctccag ctcctagacg aagtacttca |
| 2161 | taccatgccg attgcagacc cacaaccttt agactgaggt cttttaccgt tggggccctt |
| 2221 | aaccttatca ggatggtgga ctacaaaata caatcctgtt tataatctga agatatattt |
| 2281 | cacttttgtt ctgctttatc ttttcataaa gggttgaaaa tgtgtttgct gccttgctcc |
| 2341 | tagcagacag aaactggatt aaaacaattt tttttttcct cttcagaact tgtcaggcat |
| 2401 | ggctcagagc ttgaagatta ggagaaacac attcttatta attcttcacc tgttatgtat |
| 2461 | gaaggaatca ttccagtgct agaaaattta gccctttaaa acgtcttaga gccttttatc |
| 2521 | tgcagaacat cgatatgtat atcattctac agaataatcc agtattgctg attttaaagg |
| 2581 | cagagaagtt ctcaaagtta attcacctat gttattttgt gtacaagttg ttattgttga |
| 2641 | acatacttca aaaataatgt gccatgtggg tgagttaatt ttaccaagag taactttact |
| 2701 | ctgtgtttaa aaagtaagtt aataatgtat tgtaatcttt catccaaaat attttttgca |
| 2761 | agttatatta gtgaagatgg tttcaattca gattgtcttg caacttcagt tttatttttg |
| 2821 | ccaaggcaaa aaactcttaa tctgtgtgta tattgagaat cccttaaaat taccagacaa |
| 2881 | aaaaatttaa aattacgttt gttattccta gtggatgact gttgatgaag tatacttttc |
| 2941 | ccctgttaaa cagtagttgt attcttctgt atttctaggc acaaggttgg ttgctaagaa |
| 3001 | gcctataaga ggaatttctt ttccttcatt catagggaaa ggttttgtat tttttaaaac |
| 3061 | actaaaagca gcgtcactct acctaatgtc tcactgttct gcaaaggtgg caatgcttaa |
| 3121 | actaaataat gaataaactg aatattttgg aaactgctaa attctatgtt aaatactgtg |
| 3181 | cagaataatg gaaacattac agttcataat aggtagtttg gatatttttg tacttgattt |
| 3241 | gatgtgactt tttttggtat aatgtttaaa tcatgtatgt tatgatattg tttaaaattc |
| 3301 | agtttttgta tcttggggca agactgcaaa cttttttata tcttttggtt attctaagcc |
| 3361 | ctttgccatc aatgatcata tcaattggca gtgactttgt atagagaatt taagtagaaa |
| 3421 | agttgcagat gtattgactg taccacagac acaatatgta tgctttttac ctagctggta |
| 3481 | gcataaataa aactgaatct caacatacaa agttgaattc taggtttgat ttttaagatt |
| 3541 | ttttttttct tttgcacttt tgagtccaat ctcagtgatg aggtaccttc tactaaatga |
| 3601 | caggcaacag ccagttctat tgggcagctt tgtttttttc cctcacactc taccgggact |
| 3661 | tccccatgga cattgtgtat catgtgtaga gttggttttt ttttttttta atttttattt |
| 3721 | tactatagca gaaatagacc tgattatcta caagatgata aatagattgt ctacaggata |
| 3781 | aatagtatga aataaaatca aggattatct ttcagatgtg tttacttttg cctggagaac |
| 3841 | ttttagctat agaaacactt gtgtgatgat agtcctcctt atatcacctg gaatgaacac |
| 3901 | agcttctact gccttgctca gaaggtcttt taaatagacc atcctagaaa ccactgagtt |
| 3961 | tgcttatttc tgtgatttaa acatagatct tgatccaagc tacatgactt ttgtctttaa |
| 4021 | ataacttatc taccacctca tttgtactct tgattactta caaattcttt cagtaaacac |
| 4081 | ctaattttct tctgtaaaag tttggtgatt taagttttat tggcagtttt ataaaaagac |
| 4141 | atcttctcta gaaattgcta actttaggtc cattttactg tgaatgagga ataggagtga |
| 4201 | gttttagaat aacagatttt taaaaatcca gatgatttga ttaaaacctt aatcatacat |
| 4261 | tgacataatt cattgcttct tttttttgag atatggagtc ttgctgtgtt gcccaggcag |
| 4321 | gagtgcagtg gtatgatctc agctcactgc aacctctgcc tcccgggttc aactgattct |
| 4381 | cctgcctcag cctccctggt agctaggatt acaggtgccc gccaccatgc ctggctaact |
| 4441 | tttgtagttt tagtagagac ggggttttgc ctgttggcca ggctggtctt gaactcctga |
| 4501 | cctcaagtga tccatccacc ttggcctccc aaagtgctgg gattacgggc gtgagccact |
| 4561 | gtccctggcc tcattgttcc cttttctact ttaaggaaag ttttcatgtt taatcatctg |
| 4621 | gggaaagtat gtgaaaaata tttgttaaga agtatctctt tggagccaag ccacctgtct |
| 4681 | tggtttcttt ctactaagag ccataaagta tagaaatact tctagttgtt aagtgcttat |
| 4741 | atttgtacct agatttagtc acacgctttt gagaaaacat ctagtatgtt atgatcagct |
| 4801 | attcctgaga gcttggttgt taatctatat ttctatttct tagtggtagt catctttgat |
| 4861 | gaataagact aaagattctc acaggtttaa aattttatgt ctactttaag ggtaaaatta |
| 4921 | tgaggttatg gttctgggtg ggttttctct agctaattca tatctcaaag agtctcaaaa |
| 4981 | tgttgaattt cagtgcaagc tgaatgagag atgagccatg tacacccacc gtaagacctc |
| 5041 | attccatgtt tgtccagtgc ctttcagtgc attatcaaag ggaatccttc atggtgttgc |
| 5101 | ctttattttc cggggagtag atcgtgggat atagtctatc tcatttttaa tagtttaccg |
| 5161 | cccctggtat acaaagataa tgacaataaa tcactgccat ataaccttgc tttttccaga |
| 5221 | aacatggctg ttttgtattg ctgtaaccac taaataggtt gcctatacca ttcctcctgt |
| 5281 | gaacagtgca gatttacagg ttgcatggtc tggcttaagg agagccatac ttgagacatg |
| 5341 | tgagtaaact gaactcatat tagctgtgct gcatttcaga cttaaaatcc atttttgtgg |
| 5401 | ggcagggtgt ggtgtgtaaa ggggggtgtt tgtaatacaa gttgaaggca aaataaaatg |
| 5461 | tcctgtctcc cagatgatat acatcttatt atttttaaag tttattgcta attgtaggaa |
| 5521 | ggtgagttgc aggtatcttt gactatggtc atctggggaa ggaaaatttt acattttact |
| 5581 | attaatgctc cttaagtgtc tatggaggtt aaagaataaa atggtaaatg tttctgtgcc |
| 5641 | tggtttgatg gtaactggtt aatagttact caccatttta tgcagagtca cattagttca |
| 5701 | caccctttct gagagccttt tgggagaagc agttttattc tctgagtgga acagagttct |
| 5761 | ttttgttgat aatttctagt ttgctccctt cgttattgcc aactttactg gcattttatt |
| 5821 | taatgatagc agattgggaa aatggcaaat ttaggttacg gaggtaaatg agtatatgaa |
| 5881 | agcaattacc tctaaagcca gttaacaatt attttgtagg tggggtacac tcagcttaaa |
| 5941 | gtaatgcatt tttttttccc gtaaaggcag aatccatctt gttgcagata gctatctaaa |
| 6001 | taatctcata tcctcttttg caaagactac agagaatagg ctatgacaat cttgttcaag |
| 6061 | cctttccatt tttttccctg ataactaagt aatttctttg aacataccaa gaagtatgta |
| 6121 | aaaagtccat ggccttattc atccacaaag tggcatccta ggcccagcct tatccctagc |
| 6181 | agttgtccca gtgctgctag gttgcttatc ttgtttatct ggaatcactg tggagtgaaa |
| 6241 | ttttccacat catccagaat tgccttattt aagaagtaaa acgttttaat ttttagcctt |
| 6301 | tttttggtgg agttatttaa tatgtatatc agaggatata ctagatggta acatttcttt |
| 6361 | ctgtgcttgg ctatctttgt ggacttcagg ggcttctaaa acagacagga ctgtgttgcc |
| 6421 | tttactaaat ggtctgagac agctatggtt ttgaattttt agtttttttt ttttaaccca |
| 6481 | cttcccctcc tggtctcttc cctctctgat aattaccatt catatgtgag tgttagtgtg |
| 6541 | cctcctttta gcattttctt cttctctttc tgattcttca tttctgactg cctaggcaag |
| 6601 | gaaaccagat aaccaaactt actagaacgt tctttaaaac acaagtacaa actctgggac |
| 6661 | aggacccaag acactttcct gtgaagtgct gaaaaagacc tcattgtatt ggcatttgat |
| 6721 | atcagtttga tgtagcttag agtgcttcct gattcttgct gagtttcagg tagttgagat |
| 6781 | agagagaagt gagtcatatt catattttcc cccttagaat aatattttga aaggtttcat |
| 6841 | tgcttccact tgaatgctgc tcttacaaaa actggggtta caagggttac taaattagca |
| 6901 | tcagtagcca gaggcaatac cgttgtctgg aggacaccag caaacaacac acaacaaagc |
| 6961 | aaaacaaacc ttgggaaact aaggccattt gttttgtttt ggtgtcccct ttgaagccct |
| 7021 | gccttctggc cttactcctg tacagatatt tttgacctat aggtgccttt atgagaattg |
| 7081 | agggtctgac atcctgcccc aaggagtagc taaagtaatt gctagtgttt tcagggattt |
| 7141 | taacatcaga ctggaatgaa tgaatgaaac tttttgtcct ttttttttct gttttttttt |
| 7201 | ttctaatgta gtaaggacta aggaaaacct ttggtgaaga caatcatttc tctctgttga |
| 7261 | tgtggatact tttcacaccg tttatttaaa tgctttctca ataggtccag agccagtgtt |
| 7321 | cttgttcaac ctgaaagtaa tggctctggg ttgggccaga cagttgcact ctctagtttg |
| 7381 | ccctctgcca caaatttgat gtgtgacctt tgggcaagtc atttatcttc tctgggcctt |
| 7441 | agttgcctca tctgtaaaat gagggagttg gagtagatta attattccag ctctgaaatt |
| 7501 | ctaagtgacc ttggctacct tgcagcagtt ttggatttct tccttatctt tgttctgctg |
| 7561 | tttgaggggg ctttttactt atttccatgt tattcaaagg agactaggct tgatatttta |
| 7621 | ttactgttct tttatggaca aaaggttaca tagtatgccc ttaagactta attttaacca |
| 7681 | aaggcctagc accaccttag gggctgcaat aaacacttaa cgcgcgtgcg cacgcgcgcg |
| 7741 | cgcacacaca cacacacaca cacacacaca cacaggtcag agtttaaggc tttcgagtca |
| 7801 | tgacattcta gcttttgaat tgcgtgcaca cacacacgca cgcacacact ctggtcagag |
| 7861 | tttattaagg ctttcgagtc atgacattat agcttttgag ttggtgtgtg tgacaccacc |
| 7921 | ctcctaagtg gtgtgtgctt gtaatttttt ttttcagtga aaatggattg aaaacctgtt |
| 7981 | gttaatgctt agtgatatta tgctcaaaac aaggaaattc ccttgaaccg tgtcaattaa |
| 8041 | actggtttat atgactcaag aaaacaatac cagtagatga ttattaactt tattcttggc |
| 8101 | tctttttagg tccattttga ttaagtgact tttggctgga tcattcagag ctctcttcta |
| 8161 | gcctaccctt ggatgagtac aattaatgaa attcatattt tcaaggacct gggagccttc |
| 8221 | cttggggctg ggttgagggt ggggggttgg ggagtcctgg tagaggccag ctttgtggta |
| 8281 | gctggagagg aagggatgaa accagctgct gttgcaaagg ctgcttgtca ttgatagaag |
| 8341 | gactcacggg cttggattga ttaagactaa acatggagtt ggcaaacttt cttcaagtat |
| 8401 | tgagttctgt tcaatgcatt ggacatgtga tttaagggaa aagtgtgaat gcttatagat |
| 8461 | gatgaaaacc tggtgggctg cagagcccag tttagaagaa gtgagttggg ggttggggac |
| 8521 | agatttggtg gtggtatttc ccaactgttt cctcccctaa attcagagga atgcagctat |
| 8581 | gccagaagcc agagaagagc cactcgtagc ttctgctttg gggacaactg gtcagttgaa |
| 8641 | agtcccagga gttcctttgt ggctttctgt atacttttgc ctggttaaag tctgtggcta |
| 8701 | aaaaatagtc gaacctttct tgagaactct gtaacaaagt atgtttttga ttaaaagaga |
| 8761 | aagccaacta aaaaaaaaaa aaaaaaaaa |

In accordance with the present disclosure, a suitable SMAD4 inhibitor is any agent or compound capable of decreasing the level of SMAD4 expression and/or SMAD4 signaling activity in glial cells of the subject relative to the level of SMAD4 expression and/or signaling activity occurring in the absence of the agent. Suitable inhibitory agents may inhibit SMAD mRNA expression or protein expression, may block SMAD4 posttranslational processing, may inhibit SMAD4 interaction with other signaling proteins, or may block SMAD4 nuclear translocation.

In one embodiment, the SMAD4 inhibitor is a small molecule inhibitor. One exemplary SMAD4 inhibitor suitable for use in the methods disclosed herein is the deubiquitinase inhibitor, PR-619 (i.e., 2,6-Diamino-3,5-dithiocyanopyridine; CAS No. 2645-32-1) which reduces SMAD4 expression levels as described in Soji et al., “Deubiquitinase Inhibitor PR-619 Reduces Smad4 Expression and Suppresses Renal Fibrosis in Mice with Unilateral Ureteral Obstruction,” PLoS 13(8): e0202409 (2008), which is hereby incorporated by reference in its entirety. Another exemplary small molecule inhibitor of SMAD4, which also acts via decreasing SMAD4 expression and is suitable for use in the methods described herein is valproic acid (see e.g., Mao et al., “Valproic acid inhibits epithelial mesenchymal transition in renal cell carcinoma by decreasing SMAD4 expression,” Mol. Med. Rep. 16(5):6190-6199 (2017) and Lan et al., “Valproic acid (VPA) inhibits the epithelial-mesenchymal transition in prostate carcinoma via the dual suppression of SMAD4,” J Cancer Res Clin Oncol. 142(1):177-85 (2016), which are hereby incorporated by reference in their entirety). Another exemplary small molecule inhibitor of SMAD4 that is suitable for use in the methods described herein is 5-fluorouracil (5-FU), which reduces SMAD4 protein levels as taught by Okada et al., “Regulation of transforming growth factor is involved in the efficacy of combined 5-fluorouracil and interferon alpha-2b therapy of advanced hepatocellular carcinoma,” Cell Death Discov. 4:42 (2018), which is hereby incorporated by reference in its entirety. Another exemplary SMAD4 inhibitor suitable for use in the methods disclosed herein is the HDAC inhibitor vorinostat, which inhibits SMAD4 nuclear translocation as described by Sakamoto et al., “A Histone Deacetylase Inhibitor Suppresses Epithelial-Mesenchymal Transition and Attenuates Chemoresistance in Biliary Tract Cancer,” PLoS One 11(1):e0145985 (2016), which is hereby incorporated by reference in its entirety. Mitogen-activated protein kinase (MAPK)-specific inhibitors also block SMAD4 nuclear translocation as disclosed in Jiang et al., “MAPK inhibitors modulate Smad2/3/4 complex cyto-nuclear translocation in myofibroblasts via Imp?/8 mediation,” Mol Cell Biochem. 406(1-2):255-62 (2015), which is hereby incorporated by reference in its entirety). Thus, MAPK-specific inhibitors, in particular ERK, JNK, and p38-specific inhibitors, serve as an additional class of small molecule inhibitors that can be utilized in the methods disclosed herein. Suitable inhibitory agents in this class are known in the art and include, for example and without limitation, Ulixertinib (ERK inhibitor) (BVD523) (Sullivan et al., “First-in-Class ERK1/2 Inhibitor Ulixertinib (BVD-523) in Patient with MAPK Mutant Advanced Solid Tumors: Results of a Phase I Dose—Escalation and Expansion Study,” Cancer Discov. 8(2): 1-12 (2017), which is hereby incorporated by reference in its entirety); CC-401, SP600125, AS601245, AS602801, D-JNKI-1, and BI-78D (JNK inhibitors) (Cicenas et al., “JNK, p38, ERK, and SGK1 Inhibitors in Cancer,” Cancers 10: 1(2018), which is hereby incorporated by reference in its entirety); SCIO-469 (Talmapimod), BIRB-796 (Doramapimod), LY2228820 (Ralimetinib), VX-745, and PH-797804 (selective p38 inhibitor) (Cicenas et al., “JNK, p38, ERK, and SGK1 Inhibitors in Cancer,” Cancers 10: 1(2018), which is hereby incorporated by reference in its entirety).

Another class of SMAD4 inhibitors suitable for use in the methods disclosed herein include inhibitory peptides. One suitable peptide inhibitor of SMAD4 is the SBD peptide, which is capable of blocking SMAD4 protein interaction (Urata et al., “A peptide that blocks the interaction of NF-κB p65 subunit with Smad4 enhances BMP2-induced osteogenesis,” J Cell Physiol. 233(9):7356-7366 (2018), which is hereby incorporated by reference in its entirety). The SBD peptide corresponds to an amino terminal region within the transactivation of p65 that interacts with the MH1 domain of SMAD4 (called the Smad4-binding domain (SBD) (see Urata et al., “A peptide that blocks the interaction of NF-κB p65 subunit with Smad4 enhances BMP2-induced osteogenesis,” J Cell Physiol. 233(9):7356-7366 (2018), and Hirata-Tsuchiya et al., Inhibition of BMP2-Induced Bone Formation by the p65 Subunit of NK-kB via an Interaction with SMAD 4,” Mol. Endocrinology 28(9): 1460-1470 (2014), which are hereby incorporated by reference in their entirety). Binding of the SBD peptide to SMAD4 blocks SMAD4 from interacting with other proteins, such p65. An exemplary SBD peptide has the amino acid sequence of APGLPNGLLSGDEDFSSIADMDFSALLSQISS (SEQ ID NO:35).

Another suitable peptide inhibitor of SMAD4 is the coactosin-like protein (CLP or Cotl1; UniProtKB accession no. Q14019), which is an F-actin binding protein. This protein inhibits SMAD4 by causing post-transcriptional downregulation of SMAD4 (Xia et al., “Coactosin-like protein CLP/Cotl1 suppresses breast cancer growth through activation of IL-24/PERP and inhibition of non-canonical TGFβ signaling,” Oncogene 37(3):323-331 (2018), which is hereby incorporated by reference in its entirety). Accordingly, recombinant forms of CLP/Cotl1 having an amino acid sequence of SEQ ID NO: 8 (shown below), or active fragments thereof, are suitable for use in the methods disclosed herein.

(SEQ ID NO: 8)
MATKIDKEACRAAYNLVRDDGSAVIWVTFKYDGSTIVPGEQGAEYQHFIQ
QCTDDVRLFAFVRFTTGDAMSKRSKFALITWIGENVSGLQRAKTGTDKTL
VKEVVQNFAKEFVISDRKELEEDFIKSELKKAGGANYDAQTE

In another embodiment, the SMAD4 inhibitor is an inhibitory nucleic acid molecule selected from the group consisting of a SMAD4 antisense oligonucleotide, a SMAD4 shRNA, a SMAD4 siRNA, and a SMAD4 RNA aptamer.

The use of antisense methods to inhibit the in vivo translation of genes and subsequent protein expression is well known in the art (e.g., U.S. Pat. No. 7,425,544 to Dobie et al.; U.S. Pat. No. 7,307,069 to Karras et al.; U.S. Pat. No. 7,288,530 to Bennett et al.; U.S. Pat. No. 7,179,796 to Cowsert et al., which are hereby incorporated by reference in their entirety). In accordance with the present disclosure, suitable antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule encoding SMAD4 (see e.g., Weintraub, H. M., “Antisense DNA and RNA,” Scientific Am. 262:40-46 (1990), which is hereby incorporated by reference in its entirety). SEQ ID NO: 2 above is an exemplary nucleic acid molecule encoding SMAD4. Suitable antisense oligonucleotides for use in the method described herein are, or are up to 12, 13, 14, 15, 16, 17, 18, 19, 20, 21, 22, 23, 24, 25, 26, 27, 28, 29, or 30 nucleobases in length and comprise no more than 6, no more than 5, no more than 4, no more than 3, no more than 2, or no more than 1 non-complementary nucleobase(s) relative to the target SMAD4 nucleic acid, or specified portion thereof. The antisense nucleic acid molecule hybridizes to its corresponding target SMAD4 nucleic acid molecule, to form a double-stranded molecule, which interferes with translation of the mRNA, as the cell will not translate a double-stranded mRNA.

SMAD4 antisense nucleic acids can be introduced into cells as antisense oligonucleotides, or can be produced in a cell in which a nucleic acid encoding the antisense nucleic acid has been introduced, for example, using gene therapy methods. Anti-SMAD4 antisense oligonucleotides suitable for use in accordance with the methods described herein are disclosed in U.S. Pat. No. 6,013,787 to Monia et al., and Kretschmer et al., “Differential Regulation of TGF-β Signaling Through Smad2, Smad3, and Smad4,” Oncogene 22: 6748-6763 (2003), which are hereby incorporated by reference in their entirety.

SMAD4 siRNAs are double stranded synthetic RNA molecules approximately 20-25 nucleotides in length with short 2-3 nucleotide 3′ overhangs on both ends. The double stranded siRNA molecule represents the sense and anti-sense strand of a portion of the target mRNA molecule, in this case a portion of the SMAD4 nucleotide sequence, i.e., SEQ ID NO: 2 encoding SMAD4. siRNA molecules are typically designed to target a region of the SMAD4 mRNA target approximately 50-100 nucleotides downstream from the start codon. Upon introduction into a cell, the siRNA complex triggers the endogenous RNA interference (RNAi) pathway, resulting in the cleavage and degradation of the target SMAD4 mRNA molecule. siRNA molecules that target SMAD4 and other members of the SMAD4 transcription complex that can be utilized in the methods described herein are disclosed in U.S. Pat. No. 9,035,039 to Dhillon et al., and Puplampu-Dove et al., “Potentiating Tumor Immunity Using Aptamer-Targeted RNAi to Render CD8+ T Cells Resistant to TGFβ Inhibition,” J. OncoImmunology 7(4) (2018), which are hereby incorporated by reference in their entirety. Various improvements of siRNA compositions, such as the incorporation of modified nucleosides or motifs into one or both strands of the siRNA molecule to enhance stability, specificity, and efficacy, have been described and are suitable for use in accordance with this aspect of the disclosure (see e.g., WO2004/015107 to Giese et al.; WO2003/070918 to McSwiggen et al.; WO1998/39352 to Imanishi et al.; U.S. Patent Application Publication No. 2002/0068708 to Jesper et al.; U.S. Patent Application Publication No. 2002/0147332 to Kaneko et al; U.S. Patent Application Publication No. 2008/0119427 to Bhat et al., which are hereby incorporated by reference in their entirety).

Short or small hairpin RNA molecules are similar to siRNA molecules in function, but comprise longer RNA sequences that make a tight hairpin turn. shRNA is cleaved by cellular machinery into siRNA and gene expression is silenced via the cellular RNA interference pathway. shRNA molecules that effectively interfere with SMAD4 expression are described herein, and comprise the following nucleic acid sequences: 5′GUAAGUAGCUGGCUGACCA-3′ (SEQ ID NO: 3) targeting the SMAD4 nucleotide sequence of 5′-TGGTCAGCCAGCTACTTAC-3′ (SEQ ID NO: 4) and 5′-AGAAGUGAGUCAUAUUCAU-3′ (SEQ ID NO: 6) targeting the SMAD4 nucleotide sequence of 5′-ATGAATATGACTCACTTCT-3′ (SEQ ID NO: 7). Other shRNA molecules that inhibit SMAD4 expression and are suitable for use in accordance with the methods described herein are known in the art, see e.g., WO2016115558 to Doiron, which is hereby incorporated by reference in its entirety.

Nucleic acid aptamers that specifically bind to SMAD4 are also suitable for use in the methods as described herein. Nucleic acid aptamers are single-stranded, partially single-stranded, partially double-stranded, or double-stranded nucleotide sequences, capable of specifically recognizing a selected target molecule, either the SMAD4 protein having the amino acid sequence of SEQ ID NO: 1, or the SMAD4 nucleic acid molecule having the nucleotide sequence of SEQ ID NO: 2, by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides, and nucleotides comprising backbone modifications, branchpoints, and non-nucleotide residues, groups, or bridges.

Modifications to inhibitory nucleic acid molecules described herein, i.e., SMAD4 antisense oligonucleotides, siRNA, shRNA, PNA, aptamers, encompass substitutions or changes to internucleoside linkages, sugar moieties, or nucleobases. Modified inhibitory nucleic acid molecules are often preferred over native forms because of desirable properties such as, for example, enhanced cellular uptake, enhanced affinity for nucleic acid target, increased stability in the presence of nucleases, or increased inhibitory activity. For example, chemically modified nucleosides may be employed to increase the binding affinity of a shortened or truncated antisense oligonucleotide for its target nucleic acid. Consequently, comparable results can often be obtained with shorter antisense compounds that have such chemically modified nucleosides.

SMAD4 targeted inhibitory nucleic acid molecules can optionally contain one or more nucleosides wherein the sugar group has been modified. Such sugar modified nucleosides may impart enhanced nuclease stability, increased binding affinity or some other beneficial biological property to the nucleic acid molecule. In certain embodiments, nucleosides comprise a chemically modified ribofuranose ring moieties. Examples of chemically modified ribofuranose rings include without limitation, addition of substituted groups, including 5′ and 2′ substituent groups, bridging of non-geminal ring atoms to form bicyclic nucleic acids (BNA), replacement of the ribosyl ring oxygen atom with S, N(R), or C(R1)(R)2, where R═H, C1-C12 alkyl or a protecting group, and combinations thereof. Examples of chemically modified sugars include 2′-F-5′-methyl substituted nucleoside, replacement of the ribosyl ring oxygen atom with S with further substitution at the 2′-position.

In certain embodiments, nucleosides are modified by replacement of the ribosyl ring with a sugar surrogate (sometimes referred to as DNA analogs), such as a morpholino ring, a cyclohexenyl ring, a cyclohexyl ring, or a tetrahydropyranyl ring.

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to SMAD4 inhibitor nucleic acid molecules. Modified nucleobases include synthetic and natural nucleobases such as, for example, 5-methylcytosine (5-me-C). Certain nucleobase substitutions, including 5-methylcytosine substitutions, are particularly useful for increasing the binding affinity of a nucleic acid molecule to its target nucleic acid. Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, 7-methyl guanine and 7-methyladenine, 2-F-adenine, 2-aminoadenine, 8-azaguanine and 8-azaadenine, 7-deazaguanine, 7-deazaadenine, 3-deazaguanine, and 3-deazaadenine.

The naturally occurring internucleoside linkage of RNA and DNA is a 3′ to 5′ phosphodiester linkage. Inhibitory nucleic acid molecules having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparing phosphorous-containing and non-phosphorous-containing linkages are well known. In certain embodiments, an inhibitory nucleic acid molecule targeting a SMAD4 nucleic acid comprises one or more modified internucleoside linkages.

The inhibitory nucleic acid molecules described here may be covalently linked to one or more moieties or conjugates which enhance the activity, cellular distribution, or cellular uptake of the resulting inhibitory nucleic acid molecule. Typical conjugate groups include cholesterol moieties and lipid moieties. Additional conjugate groups include carbohydrates, polymers, peptides, inorganic nanostructured materials, phospholipids, biotin, phenazine, folate, phenanthridine, anthraquinone, acridine, fluoresceins, rhodamines, coumarins, and dyes.

Inhibitory nucleic acid molecule described herein can also be modified to have one or more stabilizing groups, e.g., cap structures, that are generally attached to one or both termini of the inhibitory nucleic acid molecule to enhance properties such as, for example, nuclease stability. These terminal modifications protect inhibitory nucleic acid molecules from exonuclease degradation, and can help in delivery and/or localization within a cell. Cap structures can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an inhibitory nucleic acid molecule to impart nuclease stability include those disclosed in WO 03/004602 to Manoharan, which is hereby incorporated by reference in its entirety.

In another embodiment, a suitable SMAD4 inhibitor is any agent or small molecule capable of decreasing, blocking, or preventing the interaction of SMAD4 with SMADs 2 and 3 and/or the interaction of SMAD4 with SMADs 1, 5, and 8 in a glial cell relative to the level interaction occurring in the absence of the agent.

In another embodiment, a suitable SMAD4 inhibitor is any agent or small molecule capable of antagonizing or decreasing SMAD4 activity in a glial cell relative to the level of SMAD4 activity occurring in the absence of the agent.

In one embodiment, the SMAD4 inhibitor used in accordance with the methods described herein is packaged into a nanoparticle delivery vehicle to effectuate delivery of the inhibitor to glial cells of a subject. Suitable nanoparticle delivery vehicles for delivering SMAD4 inhibitors across the blood brain barrier and/or to glial cells include, without limitation, liposome, protein nanoparticles, polymeric nanoparticles, metallic nanoparticles, and dendrimers.

Liposomes are spherical vesicles composed of phospholipid and steroid (e.g., cholesterol) bilayers that are about 80-300 nm in size. Liposomes are biodegradable with low immunogenicity. The SMAD4 inhibitor as described herein can be incorporated into liposomes using the encapsulation process. The liposomes are taken up by target cells by adsorption, fusion, endocytosis, or lipid transfer. Release of the SMAD4 inhibitor from the liposome depends on the liposome composition, pH, osmotic gradient, and surrounding environment. The liposome can be designed to release the SMAD4 inhibitor in a cell organelle specific manner to achieve, for example, nuclear delivery of the SMAD4 inhibitor.

Methods and types of liposomes that can be utilized to deliver the SMAD4 inhibitors described herein to glial cells are known in the art, see e.g., Liu et al., “Paclitaxel loaded liposomes decorated with a multifunctional tandem peptide for glioma targeting,” Biomaterials 35:4835-4847 (2014); Gao et al. “Glioma targeting and blood-brain barrier penetration by dual-targeting doxorubicin liposomes,” Biomaterials 34:5628-5639 (2013); Zong et al., “Synergistic dual-ligand doxorubicin liposomes improve targeting and therapeutic efficacy of brain glioma in animals,” Mol Pharm. 11:2346-2357 (2014); Yemisci et al., “Systemically administered brain-targeted nanoparticles transport peptides across the blood-brain barrier and provide neuroprotection,” J Cerebr Blood F Met. 35:469-475 (2015), which are hereby incorporated by reference in their entirety.

In another embodiment, the SMAD4 inhibitors described herein are packaged in a polymeric delivery vehicle. Polymeric delivery vehicles are structures that are typically about 10 to 100 nm in diameter. Suitable polymeric nanoparticles for encapsulating the SMAD4 inhibitors as described herein can be made of synthetic polymers, such as poly-ε-caprolactone, polyacrylamine, and polyacrylate, or natural polymers, such as, e.g., albumin, gelatin, or chitosan. The polymeric nanoparticles used herein can be biodegradable, e.g., poly(L-lactide) (PLA), polyglycolide (PGA), poly(lactic acid-co-glycolic acid) (PLGA), or non-biodegradable, e.g., polyurethane. The polymeric nanoparticles used herein can also contain one or more surface modifications that enhance delivery. For example, in one embodiment, the polymeric nanoparticles are coated with nonionic surfactants to reduce immunological interactions as well as intermolecular interactions. The surfaces of the polymeric nanoparticles can also be functionalized for attachment or immobilization of one or more targeting moieties as described infra, e.g., an antibody or other binding polypeptide or ligand that directs the nanoparticle across the blood brain barrier and/or to glial cells for glial cell uptake (i.e., glia progenitor or astrocyte uptake).

Methods and types of polymeric nanoparticles that can be utilized to deliver the SMAD4 inhibitors as described herein to glial cells are known in the art, see e.g., Koffie et al. “Nanoparticles enhance brain delivery of blood-brain barrier-impermeable probes for in vivo optical and magnetic resonance imaging,” Proc Natl Acad Sci USA. 108:18837-18842 (2011); Zhao et al., “The permeability of puerarin loaded poly(butylcyanoacrylate) nanoparticles coated with polysorbate 80 on the blood-brain barrier and its protective effect against cerebral ischemia/reperfusion injury,” Biol Pharm Bull. 36:1263-1270 (2013); Yemisci et al., “Systemically administered brain-targeted nanoparticles transport peptides across the blood-brain barrier and provide neuroprotection,” J Cerebr Blood F Met. 35:469-475 (2015), which are hereby incorporated by reference in their entirety.

In another embodiment, the composition of the present disclosure is packaged in a dendrimer nanocarrier delivery vehicle. Dendrimers are unique polymers with a well-defined size and structure. Exemplary nanometric molecules having dendritic structure that are suitable for use as a delivery vehicle for the SMAD4 inhibitor as described herein include, without limitation, glycogen, amylopectin, and proteoglycans. Methods of encapsulating therapeutic compositions, such as the composition described herein, in the internal structure of dendrimers are known in the art, see e.g., D'Emanuele et al., “Dendrimer-drug interactions,” Adv Drug Deliv Rev 57: 2147-2162 (2005), which is hereby incorporated by reference in its entirety. The surface of dendrimers is suitable for the attachment of one or more targeting moieties, such as antibodies or other binding proteins and/or ligands as described herein capable of targeting the dendrimers across the blood brain barrier and/or to glial cells.

An exemplary dendrimer for encapsulation of a SMAD4 inhibitor for administration and delivery to a subject in need thereof is poly(amido amide) (PAMAM). PAMAM has been utilized for the delivery of both protein and nucleic acid therapeutics to target cells of interest. Methods of encapsulating therapeutic agents in PAMAM and utilization of PAMAM for delivering therapeutic agents to the central nervous system are also known in the art and can be utilized herein, see e.g., Cerqueira et al., “Multifunctionalized CMCht/PAMAM dendrimer nanoparticles modulate the cellular uptake by astrocytes and oligodendrocytes in primary cultures of glial cells,” Macromol Biosci. 12:591-597 (2012); Nance et al., “Systemic dendrimer-drug treatment of ischemia-induced neonatal white matter injury,” J Control Release 214:112-120 (2015); Natali et al., “Dendrimers as drug carriers: dynamics of PEGylated and methotrexate-loaded dendrimers in aqueous solution,” Macromolecules 43:3011-3017 (2010); Han et al., “Peptide conjugated PAMAM for targeted doxorubicin delivery to transferrin receptor overexpressed tumors,” Mol Pharm 7: 2156-2165 (2010); Kannan et al., “Dendrimer-based Postnatal Therapy for Neuroinflammation and Cerebral Palsy in a Rabbit Model,” Sci. Transl. Med. 4:130 (2012); and Singh et al., “Folate and Folate-PEG-PAMAM dendrimers: synthesis, characterization, and targeted anticancer drug delivery potential in tumor bearing mice,” Bioconjugate Chem 19:2239-2252 (2008), which is hereby incorporated by reference in its entirety.

In another embodiment, the SMAD4 inhibitor as disclosed herein is packaged in a silver nanoparticle or an iron oxide nanoparticle. Methods and preparations of silver and iron oxide nanoparticles that can be utilized to deliver a SMAD4 inhibitor described herein to glia cells are known in the art, see e.g, Hohnholt et al., “Handling of iron oxide and silver nanoparticles by astrocytes,” Neurochem Res. 38:227-239 (2013), which is hereby incorporated by reference in its entirety.

In another embodiment, a SMAD4 inhibitor as described herein is packaged in gold nanoparticles. Gold nanoparticles are small particles (<50 nm) that enter cells via an endocytic pathway. In one embodiment, the gold nanoparticles are coated with glucose to facilitate transfer of the nanoparticles across the blood brain barrier and uptake of the nanoparticles by astrocytes via the GLUT-1 receptor as described by Gromnicova et al., “Glucose-coated Gold Nanoparticles Transfer across Human Brain Endothelium and Enter Astrocytes In vitro,” PLoS ONE 8(12): e81043 (2013), which is hereby incorporated by reference in its entirety.

In another embodiment, the composition of the present disclosure is packaged in silica nanoparticles. Silica nanoparticles are biocompatible, highly porous, and easily functionalized. Silica nanoparticles are amorphous in shape, having a size range of 10-300 nm. Silica nanoparticles that are suitable to deliver a therapeutic composition, such as a SMAD4 inhibitor to the CNS for glial cell uptake are known in the art, see e.g., Song et al., “In vitro Study of Receptor-mediated Silica Nanoparticles Delivery Across Blood Brain Barrier,” ACS Appl. Mater. Interfaces 9(24):20410-20416 (2017); Tamba et al., “Tailored Surface Silica Nanoparticles for Blood-Brain Barrier Penetration: Preparation and In vivo Investigation,” Arabian J. Chem. doi.org/10.1016/j.arabjc.2018.03.019 (2018), which are hereby incorporated by reference in their entirety.

In another embodiment, the SMAD4 inhibitor is packaged into a protein nanoparticle delivery vehicle. Protein nanoparticles are biodegradable, metabolizable, and are easily amenable to modification to allow entrapment of therapeutic molecules or compositions and attachment of targeting molecules if desired. Suitable protein nanoparticle delivery vehicles that are known in the art and have been utilized to deliver therapeutic compositions to the central nervous system include, without limitation, albumin particles (see e.g., Lin et al., “Blood-brain Barrier Penetrating Albumin Nanoparticles for Biomimetic Drug Delivery via Albumin-Binding Protein Pathway for Antiglioma Therapy,” ACS Nano 10(11): 9999-10012 (2016), and Ruan et al., “Substance P-modified Human Serum Albumin Nanoparticles Loaded with Paclitaxel for Targeted Therapy of Glioma,” Acta Pharmaceutica Sinica B 8(1): 85-96 (2018), which are hereby incorporated by reference in their entirety), gelatin nanoparticles (see e.g., Zhao et al., “Using Gelatin Nanoparticle Mediated Intranasal Delivery of Neuropeptide Substance P to Enhance Neuro-Recovery in Hemiparkinsoninan Rats,” PLoS One 11(2): e0148848 (2016), which is hereby incorporated by reference in its entirety), and lactoferrin nanoparticles (see e.g., Kumari et al., “Overcoming Blood Brain Barrier with Dual Purpose Temozolomide Loaded Lactoferrin Nanoparticles for Combating Glioma (SERP-17-12433),” Scientific Reports 7: 6602 (2017), which is hereby incorporated by reference in its entirety).

Nanoparticle mediated delivery of a therapeutic composition can be achieved passively (i.e., based on the normal distribution pattern of liposomes or nanoparticles within the body) or by actively targeting delivery. Actively targeted delivery involves modification of the delivery vehicle's natural distribution pattern by attaching a targeting moiety to the outside surface of the liposome. In one embodiment, a delivery vehicle as described herein is modified to include one or more targeting moieties, i.e., a targeting moiety that facilitates delivery of the liposome or nanoparticle across the blood brain barrier and/or a targeting moiety that facilitates glial cell uptake (i.e., glial progenitor cell uptake and/or astrocyte uptake). In one embodiment, a delivery vehicle as described herein is surface modified to express a targeting moiety suitable for achieving blood brain barrier penetration. In another embodiment, a delivery vehicle as described herein is surface modified to express a targeting moiety suitable for glial cell uptake. In another embodiment, a delivery vehicle as described herein is surface modified to express dual targeting moieties.

Targeting moieties that facilitate delivery of the liposome or nanoparticle across the blood brain barrier take advantage of receptor-mediated, transporter-mediated, or adsorptive-mediated transport across the barrier. Suitable targeting moieties for achieving blood brain barrier passage include antibodies and ligands that bind to endothelial cell surface proteins and receptors. Exemplary targeting moieties include, without limitation, cyclic RGD peptides (Liu et al, “Paclitaxel loaded liposomes decorated with a multifunctional tandem peptide for glioma targeting,” Biomaterials 35:4835-4847 (2014), which is hereby incorporated by reference in its entirety), a cyclic A7R peptide that binds to VEGFR2 and neuropilin-1 (Ying et al., “A Stabilized Peptide Ligand for Multifunctional Glioma Targeted Drug Delivery,” J. Contr. Rel. 243:86-98 (2016), which is hereby incorporated by reference in its entirety), a transferrin protein, peptide, or antibody capable of binding to the transferrin receptors (Zong et al., “Synergistic dual-ligand doxorubicin liposomes improve targeting and therapeutic efficacy of brain glioma in animals,” Mol Pharm. 11:2346-235773 (2014); Yemisci et al., “Systemically administered brain-targeted nanoparticles transport peptides across the blood-brain barrier and provide neuroprotection,” J Cerebr Blood F Met. 35:469-475 (2015); and Wei et al., “Brain Tumor-targeted Therapy by Systemic Delivery of siRNA with Transferrin Receptor-Mediated Core-Shell Nanoparticles,” Inter. J. Pharm 510(1): 394-405), Niewoehner et al., “Increased Brain Penetration and Potency of a Therapeutic Antibody Using a Monovalent Molecular Shuttle,” Neuron 81:49-60 (2014), which are hereby incorporated by reference in in their entirety), a folate protein or peptide that binds the folate receptor (Gao et al. “Glioma targeting and blood-brain barrier penetration by dual-targeting doxorubincin liposomes,” Biomaterials 34:5628-5639 (2013), which is hereby incorporated by reference in its entirety), a lactoferrin protein or peptide that binds the lactoferrin receptor (Song et al., “In vitro Study of Receptor-mediated Silica Nanoparticles Delivery Across Blood Brain Barrier,” ACS Appl. Mater. Interfaces 9(24):20410-20416 (2017), which is hereby incorporated by reference in its entirety), low density lipoprotein receptor ligands, such ApoB and ApoE (Wagner et al., “Uptake Mechanisms of ApoE-modified Nanoparticles on Brain Capillary Endothelial Cells as a Blood-brain Barrier Model,” PLoS One 7:e32568 (2012), which is hereby incorporated by reference in its entirety), substance P peptide (Ruan et al., “Substance P-modified Human Serum Albumin Nanoparticles Loaded with Paclitaxel for Targeted Therapy of Glioma,” Acta Pharmaceutica Sinica B 8(1): 85-96 (2018), which is hereby incorporated by reference in its entirety), and an angiopep-2 (An2) peptide (Demeule et al., “Conjugation of a brain-penetrant peptide with neurotensin provides antinociceptive properties,” J Clin. Invest. 124:1199-1213 (2014), which is hereby incorporated by reference in its entirety). Other suitable targeting moieties include ligands of the amino acid transporters, e.g., glutathione for transport via the glutathione transporter (Rip et al., “Glutathione PEGylated Liposomes: Pharmacokinetics and Delivery of Cargo Across the Blood-Brain Barrier in Rats,” J. Drug Target 22:460-67 (2014), which is hereby incorporated by reference in its entirety), and choline derivatives for delivery via the choline transporter (Li et al., “Choline-derivative-modified Nanoparticles for Brain-targeting Gene Delivery,” Adv. Mater. 23:4516-20 (2011), which is hereby incorporated by reference in its entirety).

A second targeting moiety is one that facilitates glial cell delivery and uptake. Suitable targeting moieties to effectuate astrocyte uptake include, without limitation, low density lipoprotein (LDL) receptor ligands or peptides thereof capable of binding the LDL receptor and oxidized LDL receptor on astrocytes (Lucarelli et al, “The Expression of Native and Oxidized LDL Receptors in Brain Microvessels is Specifically Enhanced by Astrocyte-derived Soluble Factor(s),” FEBS Letters 522(1-3): 19-23 (2002), which is hereby incorporated by reference in its entirety), glucose or other glycans capable of binding the GLUT-1 receptor on astrocytes (Gromnicova et al., “Glucose-coated Gold Nanoparticles Transfer across Human Brain Endothelium and Enter Astrocytes In vitro,” PLoS ONE 8(12): e81043 (2013), which is hereby incorporated by reference in its entirety), and platelet derived growth factor or peptide thereof capable of binding PDGFRα of glial progenitor cells.

Glial cell delivery of inhibitory nucleic acid molecules as described herein, e.g., SMAD4 antisense oligonucleotides, SMAD4 siRNA, SMAD4 shRNA, can also be achieved by packaging such nucleic acid molecules in viral vectors. Several viral vectors are known to inherently target astrocytes in vivo, e.g., lentiviral vectors (Colin et al., “Engineered Lentiviral Vector Targeting Astrocytes In vivo,” Glia 57:667-679 (2009), and Cannon et al., “Pseudotype-dependent Lentiviral Transduction of Astrocytes or Neurons in the Rat Substantia Nigra,” Exp. Neurol. 228:41-52 (2011), which are hereby incorporated by reference in their entirety), and adeno-associated virus vectors (Furman et al., “Targeting Astrocytes Ameliorates Neurologic Changes in a Mouse Model of Alzheimer's Disease,” J. Neurosci. 32: 16129-40 (2012), which is hereby incorporated by reference in its entirety), and are thus suitable for effectuating delivery of the nucleic acid SMAD4 inhibitory molecules in accordance with the methods described herein.

In one embodiment, the vector is an adenoviral-associated viral (AAV) vector. A number of therapeutic AAV vectors suitable for delivery of the nucleic acid SMAD4 inhibitors or polynucleotide encoding a SMAD4 protein inhibitor described herein to the central nervous system are known in the art. See e.g., Deverman et al., “Gene Therapy for Neurological Disorders: Progress and Prospects,” Nature Rev. 17:641-659 (2018), which in hereby incorporated by reference in its entirety. Suitable AAV vectors include serotypes AAV1, AAV2, AAV3, AAV4, AAV5, AAV6, AAV7, AAV8, AAV9, AAV10, or AAV11 in their native form or engineered for enhanced tropism. AAV vectors known to have tropism for the CNS that are particularly suited for therapeutic expression of the SMAD4 nucleic acid molecules described herein include, AAV1, AAV2, AAV4, AAV5, AAV8 and AAV9 in their native form or engineered for enhanced tropism. In one embodiment, the AAV vector is an AAV2 vector. In another embodiment, the AAV vector is an AAV5 vector as described by Vitale et al., “Anti-tau Conformational scFv MC1 Antibody Efficiently Reduces Pathological Tau Species in Adult JNPL3 Mice,” Acta Neuropathol. Commun. 6:82 (2018), optionally containing the GFAP or CAG promoter and the Woodchuck hepatitis virus (WPRE) post-translational regulatory element. In another embodiment, the AAV vector is an AAV9 vector as described by Haiyan et al., “Targeting Root Cause by Systemic scAAV9-hIDS Gene Delivery: Functional Correction and Reversal of Severe MPSII in Mice,” Mol. Ther. Methods Clin. Dev. 10:327-340 (2018), which is hereby incorporated by reference in its entirety. In another embodiment, the AAV vector is an AAVrh10 vector as described by Liu et al., “Vectored Intracerebral Immunizations with the Anti-Tau Monoclonal Antibody PHF1 Markedly Reduces Tau Pathology in Mutant Transgenic Mice,” J. Neurosci. 36(49): 12425-35 (2016), which is hereby incorporated by reference in its entirety.

In another embodiment the AAV vector is a hybrid vector comprising the genome of one serotype, e.g., AAV2, and the capsid protein of another serotype, e.g., AAV1 or AAV3-9 to control tropism. See e.g., Broekman et al., “Adeno-associated Virus Vectors Serotyped with AAV8 Capsid are More Efficient than AAV-1 or -2 Serotypes for Widespread Gene Delivery to the Neonatal Mouse Brain,” Neuroscience 138:501-510 (2006), which is hereby incorporated by reference in its entirety. In one embodiment, the AAV vector is an AAV2/8 hybrid vector as described by Ising et al., “AAV-mediated Expression of Anti-Tau ScFv Decreases Tau Accumulation in a Mouse Model of Tauopathy,” J. Exp. Med. 214(5):1227 (2017), which is hereby incorporated by reference in its entirety. In another embodiment the AAV vector is an AAV2/9 hybrid vector as described by Simon et al., “A Rapid Gene Delivery-Based Mouse Model for Early-Stage Alzheimer Disease-Type Tauopathy,” J. Neuropath. Exp. Neurol. 72(11): 1062-71 (2013), which is hereby incorporated by reference in its entirety.

In another embodiment, the AAV vector is one that has been engineered or selected for its enhanced CNS transduction after intraparenchymal administration, e.g., AAV-DJ (Grimm et al., J. Viol. 82:5887-5911 (2008), which is hereby incorporated by reference in its entirety); increased transduction of stem and progenitor cells, e.g., SCH9 and AAV4.18 (Murlidharan et al., J. Virol. 89: 3976-3987 (2015) and Ojala et al., Mol. Ther. 26:304-319 (2018), which are hereby incorporated by reference in their entirety); enhanced retrograde transduction, e.g., rAAV2-retro (Muller et al., Nat. Biotechnol. 21:1040-1046 (2003), which is hereby incorporated by reference in its entirety); or enhanced transduction of the adult CNS after IV administration, e.g., AAV-PHP.B and AAVPHP.eB (Deverman et al., Nat. Biotechnol. 34: 204-209 (2016) and Chan et al., Nat. Neurosci. 20: 1172-1179 (2017), which are hereby incorporated by reference in their entirety.

As used herein, “treating” or “treatment” includes the administration of a SMAD4 inhibitor to restore or derepress, partially or wholly, potassium channel gene expression in glial cells, restore, partially or wholly, potassium channel uptake activity in glial cells, and restore, partially or wholly, potassium homeostasis in glial cells and the surrounding tissue. With respect to treating a subject having a neuropsychiatric condition, “treating” includes any indication of success in amelioration of the condition, including any objective or subjective parameter such as abatement, remission, diminishing of symptoms (e.g., decreasing neuronal excitability), or making the condition more tolerable to the patient (e.g., decreasing seizure incident); slowing the progression of the condition; making the condition less debilitating; or improving a subject's physical or mental well-being. The treatment or amelioration of symptoms can be based on objective or subjective parameters; including the results of a physical examination, neurological examination, and/or psychiatric evaluation.

As referred to herein “under conditions effective” refers to the effective dose, route of administration, frequency of administration, formulation of SMAD4 inhibitor, etc., that play a role in achieving the desired therapeutic benefit for the subject. An effective dose of a SMAD4 inhibitor to treat a subject in accordance with the methods described herein is the dosage of SMAD4 inhibitor that derepresses potassium channel gene expression partially or wholly, which in turn will restore potassium channel uptake function (partially or wholly) to permit restoration of brain potassium homeostasis. In instances where the SMAD4 inhibitor is administered to a subject having a neuropsychiatric disorder, such as schizophrenia, an effective dose is the dose that induces glial progenitor cell differentiation to astrocytes. In another embodiment, an effective dosage is the dosage required to restore brain potassium homeostasis to a level sufficient to decrease the extracellular levels of potassium, decrease neuronal excitability, and decrease seizure incident. In another embodiment, an effective dosage to treat a subject having a neuropsychiatric disorder is the dosage effective to improve disordered cognition in the subject. The effective dose and dosing conditions for a particular subject varies, for example, depending upon the health and physical condition of the individual to be treated, the mental and emotional capacity of the individual, the stage of the disorder, the type of SMAD4 inhibitor, the route of administration, the formulation, the attending physician's assessment of the medical situation, and other relevant factors.

In one embodiment, the glial cells having impaired K⁺ channel function are glial progenitor cells. As demonstrated in the Examples herein, SMAD4 upregulation in glial progenitor cells suppresses K⁺ channel gene expression and subsequently K⁺ uptake by glial progenitor cells. The decrease in K⁺ uptake inhibits terminal glial progenitor cell differentiation. Thus, in one embodiment, an effective does of a SMAD4 inhibitor is the dose that potentiates astroglial maturation by glial progenitor cells, which reduces, eliminates, or inhibits the onset of a neuropsychiatric disease, symptoms of the neuropsychiatric disease, or side effects of a disease.

In another embodiment, the glial cells having impaired K⁺ channel function are astrocytes. SMAD4 inhibition in astrocytes restores K⁺ uptake and subsequent K⁺ homeostasis in the affected astrocytes. SMAD4 inhibition in astrocytes of a subject having a neuropsychiatric disease (where potassium channel expression and function is altered) reduces neuronal excitability, decreases seizure incidence, and improves disordered cognition. Thus, treatment with an effective dose of a SMAD4 inhibitor decreases, alleviates, arrests, or inhibits development of the symptoms or conditions associated with schizophrenia, autism spectrum disorder, bipolar disorder, or any other neuropsychiatric disorder. Treatment may be prophylactic to prevent or delay the onset or worsening of the disease, condition or disorder, or to prevent the manifestation of clinical or subclinical symptoms thereof. Alternatively, treatment may be therapeutic to suppress and/or alleviate symptoms after the manifestation of the disease, condition or disorder.

A SMAD4 inhibitor useful for restoring glial cell K⁺ uptake in a subject, for example, in a subject having a neuropsychiatric condition, may be administered by parenteral, topical, oral or intranasal means for therapeutic treatment. Intramuscular injection (for example, into the arm or leg muscles) and intravenous infusion are suitable methods of administration of the SMAD4 inhibitors disclosed herein. In some methods, such molecules are administered as a sustained release composition or device, such as a Medipad™ device (Elan Pharm. Technologies, Dublin, Ireland). Alternatively, the SMAD4 inhibitors disclosed herein are administered parenterally via intracerebral delivery, intrathecal delivery, intranasal delivery, or via direct infusion into brain ventricles.

In one embodiment, parenteral administration is by infusion. Infused SMAD4 inhibitors may be delivered with a pump. In certain embodiments, broad distribution of the infused SMAD4 inhibitor is achieved by delivery to the cerebrospinal fluid by intracranial administration, intrathecal administration, or intracerebroventricular administration.

In certain embodiments, an infused SMAD4 inhibitor is delivered directly to a tissue. Examples of such tissues include, the striatal tissue, the intracerebroventricular tissue, and the caudate tissue. Specific localization of a SMAD4 inhibitor may be achieved by direct infusion to a targeted tissue.

In certain embodiments, parenteral administration is by injection. The injection may be delivered with a syringe or a pump. In certain embodiments, the injection is a bolus administered directly to a tissue. Examples of such tissues include, the striatal tissue, the intracerebroventricular tissue, and the caudate tissue. Specific localization of pharmaceutical agents, including antisense oligonucleotides, can be achieved via injection to a targeted tissue.

In certain embodiments, specific localization of the SMAD4 inhibitor, such as a SMAD4 antisense oligonucleotide, to a targeted tissue improves the pharmacokinetic profile of the inhibitor as compared to broad diffusion of the same. The specific localization of the SMAD4 inhibitor improves potency compared to broad diffusion of the inhibitor, requiring administration of less inhibitor to achieve similar pharmacology. “Similar pharmacology” refers to the amount of time that the target SMAD4 mRNA and/or target SMAD4 protein is down-regulated/inhibited (e.g. duration of action). In certain embodiments, methods of specifically localizing a SMAD4 inhibitor, such as by bolus injection, decreases median effective concentration (EC50) of the inhibitor by a factor of about 20.

In another embodiment, the SMAD4 inhibitor as described herein is co-administered with one or more other pharmaceutical agents. According to this embodiment of the disclosure, such one or more other pharmaceutical agents are designed to treat the same disease, disorder, or condition, or one or more symptoms associated therewith, as the SMAD4 inhibitor described herein. In one embodiment, the one or more other pharmaceutical agents are designed to treat an undesired side effect of one or more pharmaceutical compositions of the present disclosure. In one embodiment, a SMAD4 inhibitor as described herein is co-administered with another pharmaceutical agent to treat an undesired effect. In another embodiment, a SMAD4 inhibitor as described herein is co-administered with another pharmaceutical agent to produce a combinational effect. In another embodiment, a SMAD4 inhibitor as described herein is co-administered with another pharmaceutical agent to produce a synergistic effect.

In one embodiment, a SMAD4 inhibitor as described herein and another pharmaceutical agent are administered at the same time. In another embodiment a SMAD4 inhibitor as described herein and another pharmaceutical agent are administered at different times. In another embodiment, a SMAD4 inhibitor as described herein and another pharmaceutical agent are prepared together in a single formulation. In another embodiment, a SMAD4 inhibitor as described herein and another pharmaceutical agent are prepared separately.

In certain embodiments, pharmaceutical agents that may be co-administered with a SMAD4 inhibitor as described herein include antipsychotic agents, such as, e.g., haloperidol, chlorpromazine, clozapine, quetapine, and olanzapine; antidepressant agents, such as, e.g., fluoxetine, sertraline hydrochloride, venlafaxine and nortriptyline; tranquilizing agents such as, e.g., benzodiazepines, clonazepam, paroxetine, venlafaxin, and beta-blockers; and mood-stabilizing agents such as, e.g., lithium, valproate, lamotrigine, and carbamazepine.

### EXAMPLES

### Materials and Methods

Patient identification, protection and sampling. Patients from which these lines were derived were diagnosed with disabling degrees of schizophrenia with onset in early adolescence; all patients and their guardians were consented/assented by a child and adolescent psychiatrist working under the supervision of one of us (RLF), and under the auspices of an approved protocol of the University Hospitals Case Medical Center Institutional Review Board, blinded as to subsequent line designations. No study investigators had access to patient identifiers.

Cell sources and lines. Schizophrenia-derived iPSC lines were produced from subjects with childhood-onset schizophrenia, and control lines were produced from age- and gender-appropriate control subjects; all iPSC lines were derived as previously reported (Windrem et al., “Human iPSC Glial Mouse Chimeras Reveal Glial Contributions to Schizophrenia,” Cell Stem Cell 21:195-208.e6 (2017), which is hereby incorporated by reference in its entirety). An additional control line (C27; Wang et al., “Human iPSC-derived Oligodendrocyte Progenitor Cells Can Myelinate and Rescue a Mouse Model of Congenital Hypomyelination,” Cell Stem Cell 12:252-264 (2013), which is hereby incorporated by reference in its entirety) was graciously provided by Dr. Lorenz Studer (Memorial Sloan-Kettering). Control-derived lines included: CWRU-22 (26 year-old male), -37 (32 year-old female), -208 (25 year-old male), and C27; SCZ-derived lines included CWRU-8 (10 year-old female), -51(16 year-old male), -52 (16 year-old male), -193 (15 year-old female), -164 (14 year-old female), -29 (12 year-old male), -30 (12 year-old male), and -31 (12 year-old male) (Windrem et al., “Human iPSC Glial Mouse Chimeras Reveal Glial Contributions to Schizophrenia,” Cell Stem Cell 21:195-208.e6 (2017), which is hereby incorporated by reference in its entirety; see Table 1). CWRU-51/52 and CWRU-29/30/31 comprised different lines from the same patients, and were assessed to estimate inter-line variability from single patients. All iPSCs were generated from fibroblasts by retroviral expression of Cre-excisable Yamanaka factors (Oct4, Sox2, Klf4, c-Myc) (Takahashi et al., “Induction of Pluripotent Stem Cells from Adult Human Fibroblasts by Defined Factors,” Cell 131:861-872 (2007), which is hereby incorporated by reference in its entirety) with validation of pluripotency and karyotypic stability as described (Windrem et al., “Human iPSC Glial Mouse Chimeras Reveal Glial Contributions to Schizophrenia,” Cell Stem Cell 21:195-208.e6 (2017), which is hereby incorporated by reference in its entirety).

TABLE 1
Patient-derived iPSC lines used in this study

| Subject | hiPSC | | | RNA-Seg | Astrocytic | Potassium | | |
|---|---|---|---|---|---|---|---|---|
| number | Line(s) | Age | Gender | of GPC s | differentiation | uptake | Karyotype | aCGH |
| | Control Subjects | | | | | | | |
| CTR 1 | 22 | 26 | M | √ | √ | √ | √ | √ |
| CTR 2 | 37 | 32 | F | √ | √ | √ | √ | √ |
| CTR 3 | 208/205 | 25 | M | √ | √ | √ | √ | √ |
| CTR 4 | C27 | NA | NA | √ | √ | √ | √ | √ |
| | Schizophrenic Subjects | | | | | | | |
| SCZ 1 | 51/52 | 16 | M | √ | √ | √ | √ | √ |
| SCZ 2 | 29, 30, | 12 | M | √ | √ | √ | √ | √ |
| | 31 | | | | | | | |
| SCZ 3 | 193 | 15 | F | | √ | √ | √ | √ |
| SCZ 4 | 164 | 14 | F | √ | √ | √ | √ | √ |
| SCZ 5 | 8 | 10 | F | √ | | | √ | √ |

The lines used in this study were previously described and published in Windrem et al., “Human iPSC Glial Mouse Chimeras Reveal Glial Contributions to Schizophrenia,” Cell Stem Cell 21:195- 208.e6 (2017), which is hereby incorporated by reference in its entirety. The additional manipulations added in the present study - astrocytic differentiation and assessment of K⁺ uptake by the resultant differentiated astrocytes - are noted in the middle columns, while karyotypic normalcy and available CGH array data are noted in the two most right-handed columns. All of these lines have normal karyotypes. CGH arrays showed that several lines have sporadic indels, but none that have been previously associated with schizophrenia or autism spectrum disorders.

hiPSC culture and passage. hiPSCs were cultured on irradiated mouse embryonic fibroblasts (MEFs), in 0.1% gelatin (Sigma G1890-100G)-coated 6-well plates with 1-1.2 million cells/well in hESC medium (see below) supplemented with 10 ng/ml bFGF (Invitrogen, 13256-029). Media changes were performed daily, and cells passaged at 80% confluence, after 4-7 days of culture. For hiPSC passage, cells were first incubated with lml collagenase (Invitrogen, 17104-019) at 37° C. for 3-5 minutes, and then cells were transferred into a 15 ml tube for centrifuge with 3 minutes. The pellet was re-suspended with ES medium with bFGF, and was plated onto new irradiated MEFs at 1:3-1:4.

GPC and astrocytic generation from hiPSCs. When hiPSCs reached 80% confluence, they were incubated with 1 ml Dispase (Invitrogen, 17105-041) to permit the generation of embryoid bodies (Ebs); these were cultured in ES medium without bFGF for 5 days. At DIVE, Ebs were plated onto poly-ornithine (Sigma, P4957) and laminin (VWR, 47743)-coated dishes, and cultured in neural induction media (NIM; see below) (Wang et al., “Human iPSC-derived Oligodendrocyte Progenitor Cells Can Myleinate and Rescue a Mouse Model of Congenital Hypomyelination,” Cell Stem Cell 12:252-264 (2013), which is hereby incorporated by reference in its entirety), supplemented with 20 ng/ml bFGF, 2 μg/ml heparin and 10 μg/ml laminin for 10 days.

At DIV 25, the Ebs were gently scraped with a 2 ml glass pipette, then cultured in NIM plus 1 μM purmorphamine (Calbiochem, 80603-730) and 0.1 μM RA (Sigma, R2625). At DIV 33, NPCs appeared and were serially switched to NIM with 1 μM purmorphamine and 10 ng/ml bFGF for 7 days, followed by glial induction medium (GIM) (Wang et al., “Human iPSC-derived Oligodendrocyte Progenitor Cells Can Myelinate and Rescue a Mouse Model of Congenital Hypomyelination,” Cell Stem Cell 12:252-264 (2013), which is hereby incorporated by reference in its entirety), with 1 μM purmorphamine for another 15 days. At DIV 56, the resultant glial spheres were mechanically cut with microsurgical blades under a dissection microscope, and switched to GIM with 10 ng/ml PDGF, 10 ng/ml IGF, and 10 ng/ml NT3, with media changes every 2 days. At DIV 80-100, CTR GPCs were cultured with 10 ng/ml BMP4 (PeproTech, AF-120-05ET) and 0.5 μM DMH1 (Sigma, D8946-5MG) for 2 weeks, and SCZ GPCs were transduced with lentiviral-SMAD4-shRNAi for 2 weeks, both of which were used for validation of K⁺ transport gene expression. At DIV 150-180, GPCs were incubated with mouse anti-CD44 microbeads (1:50), and then incubated with rabbit anti-mouse IgG2a+b micro-beads (1:100) and further sorted by magnetic cell sorting (MACS) with a magnetic stand column. The CD44⁺ cells were then matured as astrocytes in M41 supplemented with 10% FBS (VWR, 16777-014) plus 20 ng/mL BMP4 for 4 weeks.

Media recipes are listed in Table 2 (hESC and neural media) and Table 3 (Glial and Astrocyte induction media).

TABLE 2
Media formulas: Base, hESC and Neural media

| | | | |
|---|---|---|---|
| Component | Concentration | Vendor | Catalog |

hES medium

| | | | |
|---|---|---|---|
| Dulbecco's Modified Eagle | 1X | Invitrogen | 11330-032 |
| Medium/Nutrient Mixture F-12 | | | |
| KnockOut Serum Replacement | 20% | Invitrogen | 10828-028 |
| L-glutamine | 1 mM | Invitrogen | 25030-081 |
| 2-Mercaptoethanol | 0.1 mM | Sigma | M7522 |
| Non-Essential Amino Acid | 1X | Invitrogen | 11140-050 |

Neural induction medium

| | | | |
|---|---|---|---|
| Dulbecco's Modified Eagle | 1X | Invitrogen | 11330-032 |
| Medium/Nutrient Mixture F-12 | | | |
| Non-Essential Amino Acid | 1X | Invitrogen | 11140-050 |
| N2 Supplement | 1X | Invitrogen | 17502-048 |
| Recombinant human | 20 ng/ml | Sigma | F0291 |
| FGF-2 protein | | | |
| Heparin Solution | 2 μg/ml | Fisher | N00668440 |
| | | Scientific | |
| Laminin | 10 μg/ml | Fisher | CB-40232 |
| | | Scientific | |

TABLE 3
Media formulas: Glial and astrocytic induction media

| | | | |
|---|---|---|---|
| Component | Concentration | Vendor | Catalog |

Glial induction medium

| | | | |
|---|---|---|---|
| Dulbecco's Modified Eagle | 1X | Invitrogen | 11330-032 |
| Medium/Nutrient Mixture F-12 | | | |
| B27 Supplement | 1X | Invitrogen | 12587-010 |
| N1 Supplement | 1X | Sigma | N6530 |
| Non-Essential Amino Acid | 1X | Invitrogen | 11140-050 |
| Triiodo-L-Thyronine (T3) | 60 ng/ml | Sigma | T5516-1mg |
| N6,2-O-Dibutyryladenosine | 1 μM | Sigma | D0260 |
| 3′, 5′-cyclic monophosphate | | | |
| sodium salt | | | |
| Biotin | 100 ng/ml | Sigma | B4639 |
| Recombinant human PDGF- | 10 ng/ml | R&D | 221-AA-50 |
| AA protein | | | |
| Recombinant human IGF-1 | 10 ng/ml | R&D | 291-G1-050 |
| protein | | | |
| Recombinant human NT3 | 10 ng/ml | R&D | 267-N3-025 |
| protein | | | |
| Antibiotic-Antimycotic | 0.5X | Invitrogen | 15240-096 |

Astrocytic induction medium

| | | | |
|---|---|---|---|
| Dulbecco's Modified Eagle | 1X | Invitrogen | 11330-032 |
| Medium/Nutrient Mixture F-12 | | | |
| N-2-hydroxyethylpiperazine- | 1X | Invitrogen | 15630-080 |
| N-2-ethane sulfonic acid | | | |
| N1 Supplement | 1X | Sigma | N6530 |
| Non-Essential Amino Acid | 1X | Invitrogen | 11140-050 |
| D-glucose | 46.4 mM | Sigma | G8769 |
| Sodium Pyruvate | 1.5 mM | Invitrogen | 11360-070 |
| L-Glutamine | 6.35 mM | Invitrogen | 25030-081 |
| Penicillin-Streptomycin | 40 U/ml | Invitrogen | 15140-122 |
| Selenite | 0.065 μg/ml | Sigma | S9133 |
| Progesterone | 0.057 μg/ml | Sigma | P6149 |

FACS/MACS sorting. Cells were incubated with Accutase (Fisher Scientific, SCR005) for 5 minutes at 37° C. to obtain a single cell suspension, and then spun down at 200RCF for 10 minutes. These GPCs were re-suspended in cold Miltenyi Wash buffer with primary antibody (phycoerythrin (PE)-conjugated mouse anti-CD140a, 1:50, for FACS; mouse anti-CD140a, 1:100, for MACS), and incubated on ice for 30 min, gently swirling every 10 minutes. After primary antibody incubation, these cells were then washed and either incubated with a secondary antibody (rabbit anti-mouse IgG2a+b micro-beads, 1:100) followed by sorting on a magnetic stand column for MACS, or directly sorted by FACS on a FACSAria IIIu (Becton-Dickinson). The sorted cells were counted and plated onto poly-ornithine- and laminin-coated 24-well plate for further experiments. Antibodies and dilutions are listed in Table 4.

TABLE 4
Antibodies used for FACS/MACS sorting

| | | |
|---|---|---|
| REAGENT or RESOURCE | SOURCE | IDENTIFIER |

Antibodies

| | | |
|---|---|---|
| Rabbit polyclonal anti-Nanog, 1:200 | Millipore | Cat#AB9220; |
| | | RRID: AB_570613 |
| Mouse monoclonal anti-human TRA1-60, | Millipore | Cat#MA64360; |
| 1:200 | | RRID: AB_2119183 |
| Rabbit polyclonal anti-PAX6, 1:400 | Covance Research Products | Cat#PRB-278p; |
| | Inc | RRID: AB_291612 |
| Rabbit monoclonal anti-PDGF Receptor | Cell Signaling Technology | Cat#5241S; |
| alpha, clone D13C6, 1:300 | | RRID: AB_10692773 |
| Mouse monoclonal anti-human GFAP, | Covance Research Products | Cat#SMI-21R-500; RRID: |
| clone SMI 21R, 1:500 | | A6_509979 |
| Mouse monoclonal anti-S100 beta, clone | Abcam | Cat#ab11178; RRID: |
| SH-61, 1:500 | | A6_297817 |
| Goat polyclonal anti-human SOX1, 1:200 | R&D Systems | Cat#AF3369; |
| | | RRID: AB_2239897 |
| Donkey anti-mouse IgG (H + L) Alexa | ThermoFisher Scientific | Cat#A-21203; |
| Fluor 594, 1:400 | | RRID: AB_2535789 |
| Donkey anti-mouse IgG (H + L) Alexa | ThermoFisher Scientific | Cat#A-21202; |
| Fluor 488, 1:400 | | RRID: AB_141607 |
| Donkey anti-Rabbit IgG (H + L) Alexa | ThermoFisher Scientific | Cat#A-21207; |
| Fluor 594, 1:400 | | RRID: AB_141637 |
| Donkey anti-Rabbit IgG (H + L) Alexa | ThermoFisher Scientific | Cat#A-21206; |
| Fluor 488, 1:400 | | RRID: AB_2535792 |
| Donkey anti-Goat IgG (H + L) Alexa | ThermoFisher Scientific | Cat#A-11058; |
| Fluor 594, 1:400 | | RRID: AB_2534105 |
| Mouse monoclonal anti-SSEA4 FITC | Life Technologies | Cat#MC-813-70; |
| Conjugate, 1:100 | | |
| APC-conjugated mouse IgG1, Isotype | Miltenyi Biotec | Cat#130-092-214 |
| Control, 1:10 | | |
| APC-mouse IgM, Isotype Control, 1:40 | Miltenyi Biotec | Cat#130-093-176 |
| Rat anti-mouse IgG2a + b, microbeads, | Miltenyi Biotec | Cat#130-095-194 |
| 1:100 | | |
| Mouse anti-CD44 microbeads, 1:100 | Miltenyi Biotec | Cat#130-095-194 |
| APC-conjugated anti-CD133/1, 1:10 | Miltenyi Biotec | Cat#130-090-826 |
| Mouse monoclonal anti-CD140a | BD Biosciences | Cat#556001; |
| Unconjugated, 1:100 | | RRID: AB_396285 |
| PE-conjugated anti-CD140a, 1:10 | BD Biosciences | Cat#556002 |
| APC-conjugated anti-CD44, 1:10 | Miltenyi Biotec | Cat#130-095-177; |
| | | RRID: AB_10839563 |
| PE-conj. anti-mouse IgG2a, isotype | BD Pharmingen | Cat#555574 |
| control, 1:10 | | |

Chemicals, Peptides, and Recombinant Proteins

| | | |
|---|---|---|
| Dulbecco's Modified Eagle Medium | Invitrogen | Cat#11965-092 |
| Fetal Bovine Serum | Invitrogen | Cat#16000-044 |
| Non-Essential Amino Acid | Invitrogen | Cat#11140-050 |
| Dulbecco's Modified Eagle | Invitrogen | Cat#11330-032 |
| Medium/Nutrient Mixture F-12 | | |
| KnockOut Serum Replacement | Invitrogen | Cat#10828-028 |
| FBS | VWR | Cat#16777-014 |
| Donkey serum | Millipore | Cat#5058837 |
| Goat serum | Invitrogen | Cat#16210-072 |
| DPBS | Invitrogen | Cat#14190-250 |
| Thimerosal | Sigma | T5125 |
| L-glutamine | Invitrogen | Cat#25030-081 |
| Gelatin | Sigma | Cat#G1890-100G |
| 2-Mercaptoethanol | Sigma | Cat#M7522 |
| Saponin | Fluka Analytical | Cat#47036 |
| B27 Supplement | Invitrogen | Cat#12587-010 |
| N1 Supplement | Sigma | Cat#N6530 |
| Selenite | Sigma | Cat#S9133 |
| Progesterone | Sigma | Cat#P6149 |
| Rubidium-86 | PerkinElmer | Cat#NEZ072001MC |
| Ouabain | Sigma | Cat#03125 |
| Bumetanide | R&D Systems | Cat#3108 |
| Tertiapin | R&D Systems | Cat#1316 |
| NaOH | Fisher Scientific | Cat#M5X0607H6 |
| NaCl | Invitrogen | Cat#AM9760G |
| KCl | Invitrogen | Cat#AM9640G |
| CaCl₂ | Sigma | Cat#21108-500g |
| vitamin C | Sigma | Cat#A4034-100G |
| NaHCO₃ | Sigma | Cat#S-8875 |
| MgCl₂ | Sigma | Cat#M8266-IKG |
| NaH₂PO₄ | Sigma | Cat#53264-500G |
| Glucose | Sigma | Cat#G8769-100ML |
| Cocktail liquid | Fisher Scientific | Cat#509050575 |
| N2 Supplement | Invitrogen | Cat#17502-048 |
| bFGF | Sigma | Cat#F0291 |
| bFGF | Invitrogen | Cat#13256-029 |
| Collagenase | Invitrogen | Cat#17104-019 |
| Dispase | Invitrogen | Cat#17105-041 |
| Poly-ornithine | Sigma | Cat#P4957 |
| Laminin | VWR | Cat#47743 |
| Biotin | Sigma | Cat#134639 |
| dibutyryl cAMP | Sigma | Cat#D0260 |
| Heparin | Fisher | Cat#NC9484621 |
| IGF-1 | R&D Systems | Cat#291-G1-050 |
| Laminin | Corning | Cat#354232 |
| NT3 | R&D Systems | Cat#267-N3-025 |
| PDGFaa | R&D Systems | Cat#221-AA-50 |
| BMP4 | PeproTech | Cat#AF-120-05ET |
| Accutase | Fisher Scientific | Cat#SCR005 |
| Purmorphamine | Calbiochem | Cat#80603-730 |
| Retinoic acid | Sigma | Cat#R2625 |
| DMH1 | Sigma | Cat#D8946-SMG |
| T3 | Sigma | Cat#T5516-1MG |
| 4% paraformaldehyde | Fisher Scientific | Cat#NC9245948 |
| X-tremeGENE | Roche | Cat#06366236001 |
| Doxycycline | Fisher Scientific | Cat#ICN19895510 |

Critical Commercial Assays

| | | |
|---|---|---|
| RNeasy mini kit | QIAGEN | Cat#74104 |
| QIAamp DNA micro kit | QIAGEN | Cat#56304 |
| Taqman Reverse Transcription kit | Fisher Scientific | Cat#N8080234 |
| BCA Protein Assay kit | Fisher Scientific | Cat#23227 |
| Perkin Elmer LLC ULTIMA-GOLD LITERS | Fisher Scientific | Cat#509050575 |

Deposited Data

| | | |
|---|---|---|
| All raw data | Mendeley data | doi:10.17632/wynxgw7xzfl |
| RNA expression data | GEO | accession no. G5E86906 |
| Data processing and analytic routines | Github | https://github.com/cbtneph/ |
| | | GoldmanetalSCZ2016 |

Experimental Models: Cell Lines

| | | |
|---|---|---|
| C27 | L. Studer, SKI | N/A |
| CWRU8, female, age 10 | P. Tesar, Case Western | N/A |
| CWRU208, male, age 25 | P. Tesar, Case Western | N/A |
| CWRU22, male, age 26 | P. Tesar, Case Western | N/A |
| CWRU29, male, age 12 (same person as | P. Tesar, Case Western | N/A |
| line 30 and 31) | | |
| CWRU30, male, age 12 (same person as | P. Tesar, Case Western | N/A |
| line 29 and 31) | | |
| CWRU31, male, age 12 (same person as | P. Tesar, Case Western | N/A |
| line 29 and 30) | | |
| CWRU37, female, age 32 | P. Tesar, Case Western | N/A |
| CWRU51 male, age 16 (same person as | P. Tesar, Case Western | N/A |
| line 52) | | |
| CWRU52 male, age 16 (same person as | P. Tesar, Case Western | N/A |
| line 51) | | |
| CWRU164, female, age 14 | P. Tesar, Case Western | N/A |
| CWRU193, female, age 15 | P. Tesar, Case Western | N/A |
| 293T | Fisher Scientific | Cat#R70007 |

Oligonucleotides

| | | |
|---|---|---|
| ShRNA targeting sequence: SMAD4 #1: | GE Healthcare | Cat#V35H11252 |
| TGGTCAGCCAGCTACTTAC (SEQ ID NO: 4); | | |
| 2: ATGAATATGACTCACTTCT (SEQ ID NO: 7) | | |
| shScramble: | This paper | N/A |
| AAGTTGCAAATCGCGTCTCTA (SEQ ID NO: 5) | | |

Recombinant DNA

| | | |
|---|---|---|
| human cDNA of SMAD4 | GE Healthcare | Cat#MH56278 |
| Plasmid: pTANK-EF1a-coGFP-P2a-Puro- | This paper | N/A |
| WPRE | | |
| Plasmid: pTANK-EF1a-IRES-mCherry- | Benraiss et al., 2016 | N/A |
| WPRE | | |

Bacterial and Virus Strains

| | | |
|---|---|---|
| TOP10 Chemically Competent E.coli | Invitrogen | Cat#K4600-01 |

Software and Algorithms

| | | |
|---|---|---|
| Photoshop C56 | Adobe | N/A |
| Illustrator C56 | Adobe | N/A |
| FlowJo | TreeStar | N/A |
| Ingenuity Pathway Analysis | QIAGEN | https:// |
| | | www.qiagenbioinformatics.com/ |
| | | products/ingenuity-pathway- |
| | | analysis/ |
| TRANSFAC | Genexplain | https:/www.genexplain.com/ |
| | | transfac/ |
| minfi (version 1.28.2) | (Aryee et al., “Minfi: a | https://bioconductor.org/ |
| | Flexible and Comprehensive | packages/release/bioc/html/ |
| | Bioconductor Package for the | minfi.html |
| | Analysis of Infinium DNA | |
| | Methylation Microarrays,” | |
| | Bioinformatics 30:1363-1369 | |
| | (2014), which is hereby | |
| | incorporated by reference in | |
| | its entirety | |

Other

| | | |
|---|---|---|
| Agilent Bioanalyzer | Agilent | N/A |
| BD FACS Aria IIIU | BD Biosciences | N/A |
| Ultracentrifuge | Beckman | Cat#L8-70 |
| Becksman Coulter | Beckman | Cat#LS6500 |
| Hemocytometer | Fisher Scientific | Cat#02-671-54 |
| HiSeq 2500 | Illumina | N/A |
| Nanodrop 1000 spectrophotometer | Nanodrop | N/A |
| Olympus IX71 Inverted Microscope | Olympus | N/A |
| QuantStudio 12K Flex Real-Time PCR | Applied Biosystems | N/A |
| system | | |
| Orca-R2 Digital CCD Camera | Hamamatsu | Cat#C10600-10B |

RT-PCR. Total RNA was extracted from cell lines with miRNeasy mini kit (Qiagen, 217004), and then was reversely transcribed into cDNA with Taqman Reverse Transcription kit (Fisher Scientific, N8080234). The relative expression of mRNA was measured by the Bio-RAD 56048, which was further normalized to the expression of 18S mRNA.

The primer sequences are listed in Table 5.

TABLE 5
RT-PCR Primers

| Target | Forward primer | Reverse primer | Accession no. |
|---|---|---|---|
| 18S | CTGGATACCGCAGCTAGGAA | CCCTCTTAATCATGGCCTCA | NT_167214 |
| | (SEQ ID NO: 9) | (SEQ ID NO: 10) | |
| GFAP | TGCGGCCGATTGTGAAC | CCTCTTTTCTCTGCGGAACG | NM_001193376.1 |
| | (SEQ ID NO: 11) | T (SEQ ID NO: 12) | |
| BMPR2 | CTACCATGGACCATCCTGCT | CCTATCCCAAGGTCTTGCTG | NM_001204.6 |
| | (SEQ ID NO: 13) | (SEQ ID NO: 14) | |
| RUNX2 | GTGGACGAGGCAAGAGTTTC | TTCCCGAGGTCCATCTACTG | NM_001015051.3 |
| | (SEQ ID NO: 15) | (SEQ ID NO: 16) | |
| BMP1 | TCAGGAACCTCACCTTGGAC | GCACAGTGGGGAGAAGAGAG | NM_001199.3 |
| | (SEQ ID NO: 17) | (SEQ ID NO: 18) | |
| SERPINE1 | GATTGATGACAAGGGCATGG | CCCATAGGGTGAGAAAACCA | NM_000602.4 |
| | (SEQ ID NO: 19) | (SEQ ID NO: 20) | |
| BAMBI | ATCGCCACTCCAGCTACATC | GGCAGCATCACAGTAGCATC | NM_012342.2 |
| | (SEQ ID NO: 21) | (SEQ ID NO: 22) | |
| SMAD4 | CCATTTCCAATCATCCTGCT | ACCTTTGCCTATGTGCAACC | NM_005359 |
| | (SEQ ID NO: 23) | (SEQ ID NO: 24) | |
| FST | GGAGGACGTGAATGACAACA | CACGTTCTCACACGTTTCTT | BC004107.2 |
| | (SEQ ID NO: 25) | TAC (SEQ ID NO: 26) | |
| GREM1 | GGCCAGTGCAACTCTTTCTA | CTGTAGTTCAGGGCAGTTGA | AF110137.2 |
| | (SEQ ID NO: 27) | G (SEQ ID NO: 28) | |
| KCNJ9 | GTTATCCTCGAGGGCATGGT | CGTCCTCCAGAGTCAGCACT | NM_004983.2 |
| | (SEQ ID NO: 29) | (SEQ ID NO: 30) | |
| SLC12A6 | AACTGTTAGACGACGGACAT | CTTCGGTCTGGTGTCCATTT | NM_001042497.1 |
| | AG (SEQ ID NO: 31) | (SEQ ID NO: 32) | |
| ATP1A2 | TGAACCATCCAACGACAATC | CTTGCTGAGGTACCATGTTC | NM_000702.3 |
| | TA (SEQ ID NO: 33) | T (SEQ ID NO: 34) | |

In vitro immunocytochemistry. Cells were first fixed with 4% paraformaldehyde for 5 minutes at room temperature. After washing with D-PBS (Invitrogen, 14190-250) with thimerosal (Sigma, T5125) for 3 times, cells were penetrated with 0.1% saponin (Fluka Analytical, 47036) plus 1% of either goat or donkey serum for 15 minutes at room temperature. Cells were further blocked with 5% of either goat or donkey serum plus 0.05% saponin for 15 minutes at RT. After incubation with primary antibodies at 4° C. overnight, the cells were incubated with secondary antibodies for 30 min at RT. The counts of immunofluorescent cells were taken from 10 random fields per each replicate, and each sample had three replicates. Antibodies and dilutions used see Table 4.

Methylation. DNA was extracted from iPSC lines with the QIAamp DNA micro kit (Qiagen, 56304), and then whole genome methylation analysis was performed using Illumina Methylation Epic arrays; this was done at the UCLA Neuroscience Genomics Core. Raw data from Intensity Data (IDAT) files were imported into R and normalized with the preprocessQuantile function from the package minfi (Aryee et al., “Minfi: a Flexible and Comprehensive Bioconductor Package for the Analysis of Infinium DNA Methylation Microarrays,” Bioinformatics 30:136301369 (2014), which is hereby incorporated by reference in its entirety). Probes with poor quality signal were eliminated based on set threshold of detection p values (>0.01). Probes were also eliminated if they map to the sex chromosomes, to multiple genomic locations, or if they contain single nucleotide polymorphisms at the CpG site. Following preprocessing, samples were assessed by principal component analysis based on their features of methylated intensities (M-values). To determine if a covariate (sex, age, cell line, etc.) could explain variation in the samples' methylation landscape, a linear regression model was fit for covariates and each principal component. Covariates with significant p values (<0.05) were highlighted, indicating meaningful relationship between changes in the covariate (predictor variable) and changes in the principal component values (response variable).

Molecular cloning and viral construction. The human cDNA encoding SMAD4 (GE Healthcare, MHS6278) was cloned downstream of the EFla promoter in pTANK-EF1a-IRES-mCherry-WPRE (Benraiss et al., “Human Glia Can Both Induce and Rescue Aspects of Disease Phenotype in Huntington Disease,” Nature Communications 7:11758 (2016), which is hereby incorporated by reference in its entirety). The lentiviral vector allowed for expression of SMAD4 in tandem with the reporter mCherry. SMAD4 Doxycycline-inducible shRNAs of human SMAD4 (Gene target sequence: TGGTCAGCCAGCTACTTAC (SEQ ID NO:4) or ATGAATATGACTCACTTCT (SEQ ID NO:7)) in pSMART-TRE3G-EGFP-Puro-WPRE were ordered from GE Healthcare (V3SH11252). BAMBI The human shRNA and cDNA of BAMBI were generated previously (Sim et al., “Complementary Patterns of Gene Expression by Human Oligodendrocyte Progenitors and Their Environment Predict Determinants of Progenitor Maintenance and Differentiation,” Ann. Neurol. 59:763-779 (2006), which is hereby incorporated by reference in its entirety). The final constructs were validated for the correct insertion by sequencing. The plasmids were then co-transfected with pLP-VSV (Invitrogen, K497500) and psPAX2 (a gift from Didier Trono, Addgene 12260) into 293FT cells (Fisher Scientific, R70007) through X-tremeGENE (Roche, 06366236001) for lentiviral generation. The supernatants of 293T cells were then collected and spun at 76000 RCF for 3 hours to concentrate virus (Beckman L8-70, Ultracentrifuge). A 10-fold serial dilution of virus was then prepared and transduced into 293T cells, and fluorescent colonies counted to estimate viral titer.

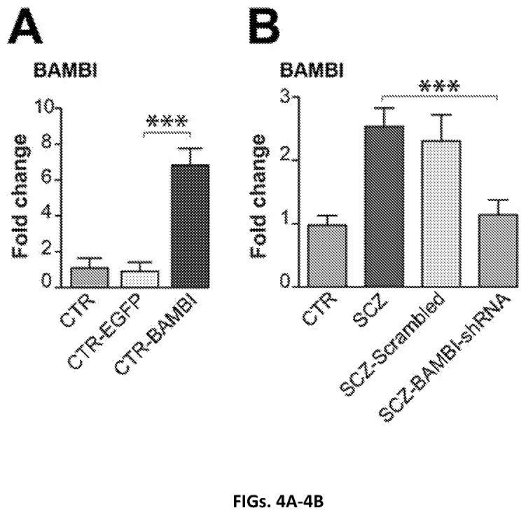

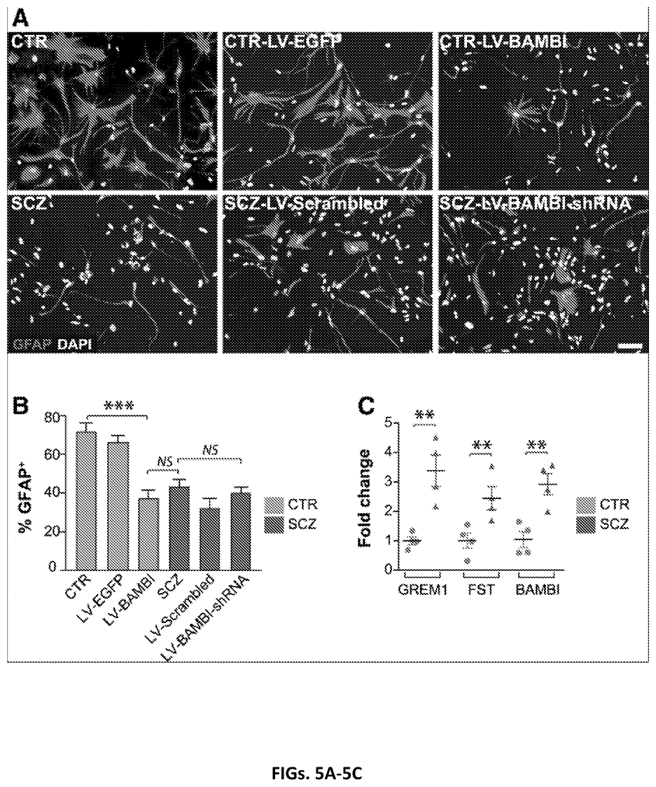

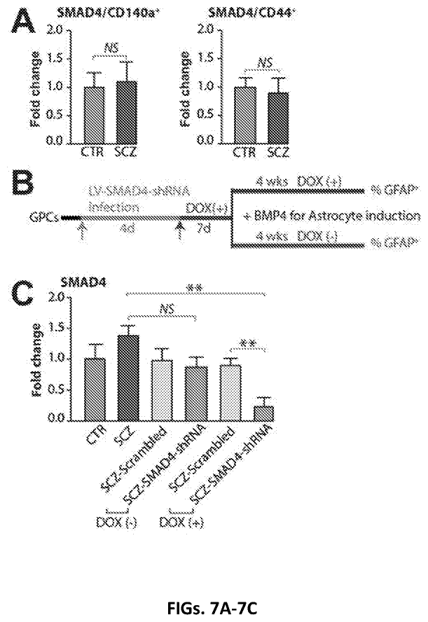

Cell transduction. CD140a⁺ hGPCs were isolated by MACS and then transduced with either lenti-TRE3G-SMAD4-shRNAi or lenti-EF1α-BAMBI-shRNAi, or their respective scrambled control viruses. Lenti-EF1α-BAMBI-shRNAi efficiently inhibited the expression of target genes (FIG. 4B). Cells infected with lenti-TRE3G-SMAD4-shRNAi were treated with 0.5 μg/ml doxycycline (Fisher, CN19895510) beginning 4 days after viral infection; this was maintained for 1 week prior to experiment initiation; during this period, the cells were maintained in glial induction media. Under doxycycline, SMAD4 mRNA expression fell to <30% of control; no inhibition was noted in the absence of doxycycline (FIGS. 7A-7C).

Potassium uptake. Astrocytes were plated onto poly-ornithine- and laminin-coated 24-well plates with 30,000 cells/well. For the potassium uptake assay, astrocytes were incubated with ⁸⁶Rb (1.0-3.3 μCi/well) for 15 minutes, and then they were washed three time with ice-cold artificial cerebrospinal fluid (aCSF, 500 μL/well). 0.5N NaOH (200 μL/well) was put into each well for cell lysis, which was put into 5 ml cocktail liquid (Ultima Gold, Fisher Scientific, 509050575) and measured by scintillation counter (Beckman Coulter, LS6500), and the results were normalized to both total protein (BCA Protein Assay Kit, Fisher Scientific, 23227) and cell number (Hemocytometer, Fisher Scientific, 02-671-54). The aCSF solution contained (in mM): 124 NaCl, 2.5 KCl, 1.75 NaH₂PO₄, 2 MgCl₂, 2 CaCl₂, 0.04 vitamin C, 10 glucose and 26 NaHCO₃, pH 7.4.

Quantification and Statistical analysis. Statistical parameters including the exact n, the center, dispersion, precision measures (mean±SEM), and statistical significance are reported in the Figures and Figure Legends. All analyses were done with GraphPad PRISM 6 using one-way ANOVA and two tailed t-test. Statistical significance was considered as P-values less than 0.05. Significances were represented as *p<0.05, **p<0.01 and ***p<0.001. Graphs and figures were made and assembled with Prism 6.

### Example 1—Astrocytic Differentiation was Impaired in SCZ GPCs

iPSCs were produced from skin samples obtained from patients with childhood-onset schizophrenia, as well as healthy young adult controls free of known mental illness, as previously described (Windrem et al., “Human iPSC Glial Mouse Chimeras Reveal Glial Contributions to Schizophrenia,” Cell Stem Cell 21:195-208.e6 (2017), which is hereby incorporated by reference in its entirety). Patient identifiers were not available to investigators besides the treating psychiatrist, although age, gender, race, diagnosis and medication history accompanied cell line identifiers. Briefly, fibroblasts were isolated from each sample; from these, 8 hiPSC lines were derived from patient samples and normal controls (5 juvenile-onset schizophrenia patients and 3 healthy gender-matched and age-analogous controls (Table 1). iPSCs were generated using excisable floxed polycistronic hSTEMCCA lentivirus (Somers et al., “Generation of Transgene-free Lung Disease-specific Human Induced Pluripotent Stem Cells Using a Single Excisable Lentiviral Stem Cell Cassette,” Stem Cells 28:1728-1740 (2010); Zou et al., “Establishment of Transgene-free Induced Pluripotent Stem Cells Reprogrammed from Human Stem Cells of Apical Papilla for Neural Differentiation,” Stem Cell Res Ther 3:43 (2012), which are hereby incorporated by reference in their entirety) encoding Oct4, Sox2, Klf4 and c-Myc (Takahashi et al., “Induction of Pluripotent Stem Cells from Adult Human Fibroblasts by Defined Factors,” Cell 131:861-872 (2007); Welstead et al., “Generating iPS Cells from MEFS Through Forced Expression of Sox-2, Oct-4, c-Myc, and Klf4,” J. Vis. Exp. 14:734 (2008), which are hereby incorporated by reference in their entirety)). A fourth hiPSC control line, C27 (Chambers et al., “Highly Efficient Neural Conversion of Human ES and iPS Cells by Dual Inhibition of SMAD Signaling,” Nature Biotechnol. 27:275-280 (2009), which is hereby incorporated by reference in its entirety), was also used, to ensure that all genomic and phenotypic data were consistent with prior work (Wang et al., “Human iPSC-derived Oligodendrocyte Progenitor Cells Can Myleinate and Rescue a Mouse Model of Congenital Hypomyelination,” Cell Stem Cell 12:252-264 (2013), which is hereby incorporated by reference in its entirety). All lines were validated as pluripotent using RNA sequencing and immunolabeling to assess pluripotent gene expression. The identity of each iPSC line was confirmed to match the parental donor fibroblasts using short tandem repeat (STR)-based DNA fingerprinting, and each line was karyotyped and arrayed for comparative genomic hybridization to confirm genomic integrity. In addition, these iPSC lines were arrayed for genome-wide methylation to compare their methylation state.

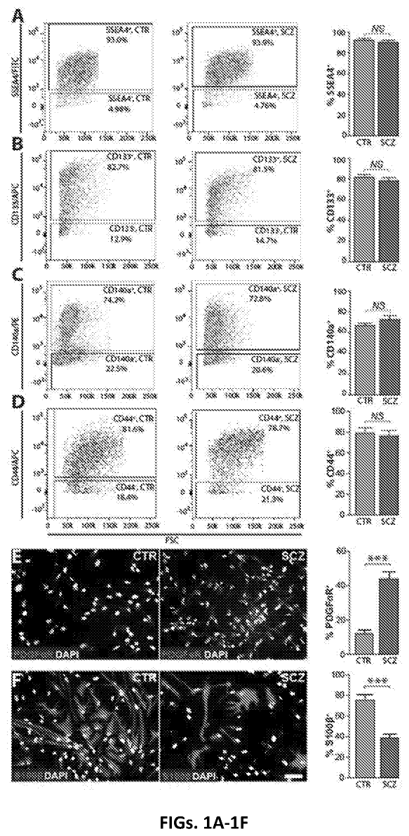

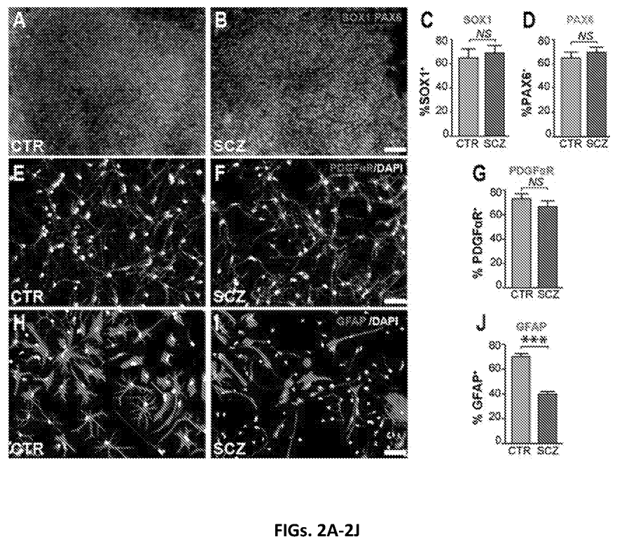

The glial differentiation efficiency of cells derived from SCZ patients and control subjects (n=4 lines from 4 different patients, each with ≥3 repeats/patient, each versus paired control) was first compared, by instructing these iPSC cells to GPC fate as previously described (Wang et al., “Human iPSC-derived Oligodendrocyte Progenitor Cells Can Myleinate and Rescue a Mouse Model of Congenital Hypomyelination,” Cell Stem Cell 12:252-264 (2013), which is hereby incorporated by reference in its entirety), and assessing their expression of stage-specific markers of maturation as a function of time. It was found that all tested iPSCs exhibited typical colonies, and expressed markers of pluripotency by flow cytometry, including SSEA4 (FIG. 1A). At the neural progenitor cell (NPC) stage, both ICC and flow cytometry revealed that the expression levels of the stage-selective markers paired box protein pax-6 (PAX6), sex determining region Y-box 1 (SOX1) and the cell surface marker prominin-1/CD133, were no different between CTR- and SCZ-derived lines (FIGS. 2A-2D; FIG. 1B). At the GPC stage, their expression of the GPC-selective platelet-derived growth factor receptor alpha (PDGFRa/CD140a) (Sim et al., “CD140a Identifies a Population of Highly Myelinogenic, Migration-Competent and Efficiently Engrafting Human Oligodendrocyte Progenitor Cells,” Nature Biotechnol. 29:934-941 (2011), which is hereby incorporated by reference in its entirety) was then assessed, which revealed that the efficiencies of GPC generation did not differ significantly between SCZ- and CTR-derived NPCs (FIGS. 2E-2G; FIG. 1C). At the astrocytic progenitor stage, the flow cytometry confirmed that the expression levels of cell surface marker CD44 was no different between CTR- and SCZ-derived lines (FIG. 1D). Thus, no differences in the differentiation of SCZ and CTR iPSCs were noted through the GPC and astrocytic progenitor stages.

At that point, the SCZ- and CTR-derived GPCs were further differentiated into astrocytes, by incubating in M41 medium supplemented with 20 ng/ml BMP4 for 4 weeks. Immunolabeling revealed that the proportion of GFAP⁺ astrocytes was significantly higher in control lines (4 CTR lines, n≥3 per line, mean of 4 CTR lines=70.1±2.4%) than in SCZ lines (4 SCZ lines, n≥3 per line, mean of 4 SCZ lines=39.9±2.0%; p<0.001, 2-tailed t-test) (FIGS. 2H-2J). In addition to GFAP, the percentage of S100β⁺ astrocytes was also significantly higher in CTR lines relative to SCZ lines (FIG. 1F). In contrast, the proportion of PDGFαR⁺ GPCs was significantly higher in BMP4-treated SCZ glia (4 SCZ lines, n≥3 per line) relative to BMP4-treated CTR glia (4 CTR lines, n≥3 per line) (FIG. 1E). This defect of astrocytic differentiation was consistently observed in all SCZ GPCs relative to CTR cells, and comprised an in vitro correlate to previously described astroglial differentiation defects in vivo (Windrem et al., “Human iPSC Glial Mouse Chimeras Reveal Glial Contributions to Schizophrenia,” Cell Stem Cell 21:195-208.e6 (2017), which is hereby incorporated by reference in its entirety).

### Example 2—SCZ GPCs Upregulated Expression of BAMBI, an Inhibitor of BMP Signaling

To identify the molecular concomitants to the defective astrocytic differentiation of SCZ GPCs, RNA-seq was earlier performed on FACS-sorted CD140a⁺ GPCs from 3 different CTR- and 4 SCZ-derived lines at time points ranging from 154 to 242 days in vitro (Windrem et al., “Human iPSC Glial Mouse Chimeras Reveal Glial Contributions to Schizophrenia,” Cell Stem Cell 21:195-208.e6 (2017), which is hereby incorporated by reference in its entirety). mRNA was isolated from these cells with polyA-selection for RNA sequencing on an Illumina HiSeq 2500 platform for approximately 45 million 1×100 bp reads per sample. The original counts were analyzed to determine disease-dysregulated genes at 5% FDR and log2 fold change >1. By that means, 118 mRNAs were identified that were consistently and significantly differentially expressed by CD140a-sorted SCZ hGPCs relative to their control iPSC hGPCs (Windrem et al., “Human iPSC Glial Mouse Chimeras Reveal Glial Contributions to Schizophrenia,” Cell Stem Cell 21:195-208.e6 (2017), which is hereby incorporated by reference in its entirety). Among these, a number of genes involved in glial lineage progression were downregulated in SCZ hGPCs, relative to their normal controls, suggesting that astroglial differentiation was impaired in SCZ in a cell-autonomous fashion, due to intrinsic defects in SCZ-derived glial progenitor cells.

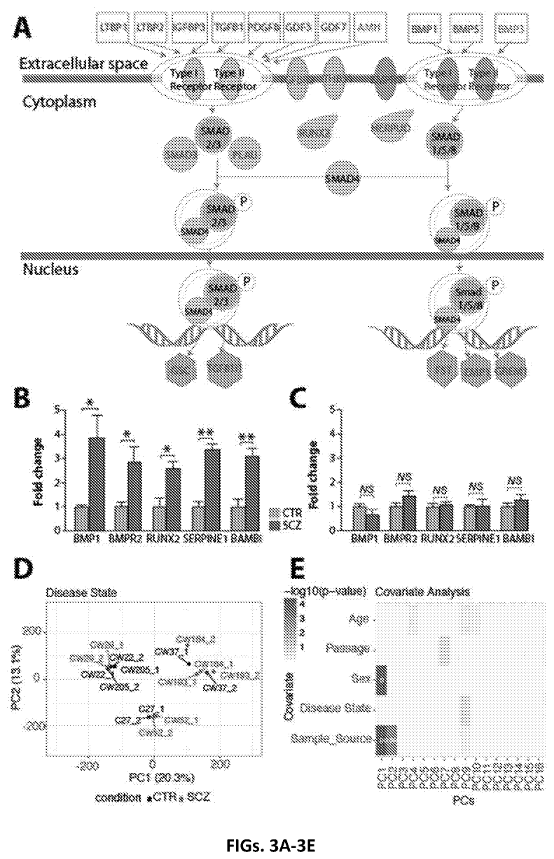

Capitalizing upon these earlier data, in this study Ingenuity Pathway Analysis (IPA) was first used to identify pathways that were significantly differentially regulated in SCZ hGPCs. It was found that among these, BMP signaling-related transcripts were upregulated in SCZ hGPCs, compared to CTR hGPCs (FIG. 3A). qPCR then validated that the expression of a number of TGFβ pathway regulators, including BAMBI, was indeed significantly elevated in SCZ GPCs (FIG. 3B). In contrast, these BMP signaling-related transcripts did not differ between SCZ and CTR lines at the NPC stage (FIG. 3C). Moreover, the methylation states of CTR- and SCZ-derived iPSCs were similar (FIG. 3D); the little variability noted across lines in iPSC methylation state appeared due to sex and line, but not to disease state or subject age (FIG. 3E). Thus, the upregulation of BAMBI and other TGFβ and BMP pathway regulators that were noted in SCZ hGPCs was not due to any systematic, disease-dependent difference in methylation pattern between CTR and SCZ cells at the pluripotent stem cell stage.

BMP4 is a strong stimulus for astrocytic differentiation by human GPCs, and BAMBI is a strong antagonist to BMP4-induced glial induction, acting as a pseudo-receptor and hence dominant-negative inhibitor of BMP signaling (Sim et al., “Complementary Patterns of Gene Expression by Human Oligodendrocyte Progenitors and Their Environment Predict Determinants of Progenitor Maintenance and Differentiation,” Ann Neurol 59:763-779 (2006), which is hereby incorporated by reference in its entirety). Yet BAMBI expression may be activated by TGFβ and BMP receptor-dependent signaling, as a compensatory negative feedback response (Onichtchouk et al., “Silencing of TGF-beta Signaling by the Pseudoreceptor BAMBI,” Nature 40:480-485 (1999), which is hereby incorporated by reference in its entirety). Accordingly, the RNA-seq, qPCR, data revealed that both BMP signaling-dependent transcripts and BAMBI were upregulated in SCZ hGPCs, but not in SCZ hNPCs (FIGS. 3B-3C). These data suggest that the upregulation of BMP signaling was specific to SCZ glia and first appeared at the glial progenitor stage, and that this process was associated with the upregulated expression of BAMBI, which in turn suppressed the astrocytic differentiation of SCZ hGPCs.

On that basis, it was asked whether BAMBI over-expression in normal control subject-derived hGPCs might mimic or reproduce the SCZ GPC phenotype, by suppressing the differentiation of these hGPCs. To that end, the expression of BAMBI was genetically modulated in hGPCs, both SCZ and CTR-derived GPCs (FIGS. 4A-4B). It was found that overexpression of BAMBI in CTR GPCs significantly decreased their efficiency of astrocytic transition (4 CTR lines with 3 repeats/each line, means of 4 CTR lines/36.4%±4.3%), yielding cells that resembled SCZ hGPCs in their refractoriness to terminal astrocytic maturation (4 SCZ lines with 3 repeats/each line, means of 4 SCZ lines/45.5%±3.6%; p=0.12 by two-tailed t test) (FIGS. 5A-5B). However, BAMBI knockdown in SCZ GPCs did not rescue astrocytic differentiation in the latter, suggesting that BAMBI overexpression contributed to the resistance of SCZ hGPCs to maturation, but was not sufficient in this regard (FIGS. 5A-5B). Accordingly, when qPCR was used to assess the expression of alternative inhibitors of BMP signaling, it was found that the mRNAs encoding both follistatin (FST) and gremlin1 (GREM1), two potent antagonists of BMPs and BMP-dependent signaling, were both significantly upregulated by SCZ GPCs (SCZ vs CTR; 4 SCZ and 4 CTR lines, 3 repeats/each line; ddCt of FST=2.45±0.39, p<0.05; GREM1=3.38±0.53, p<0.01; two-tailed t test) (FIG. 5C).

### Example 3—Astrocytic Differentiation by SCZ GPCs May be Rescued by SMAD4 Knockdown

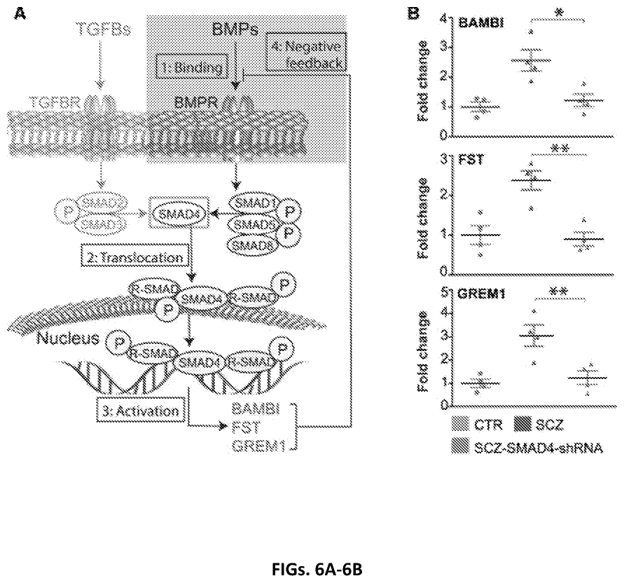

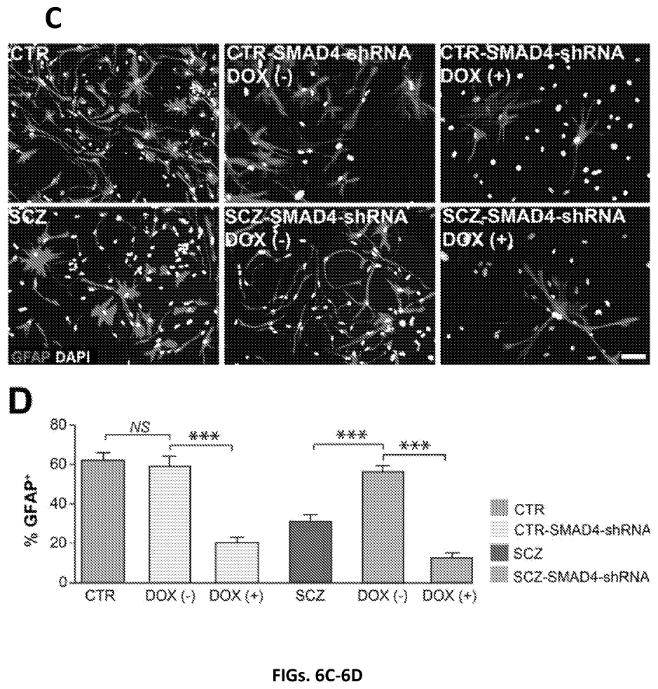

SMAD4 is necessary for canonical BMP signaling, in that it acts as a common effector for multiple upstream signals, in response to which it translocates to the nucleus, where it activates both BMP and TGFB-regulated genes (Herhaus and Sapkota, “The Emerging Roles of Deubiquitylating Enzymes (DUBS) in the TGFbeta and BMP Pathways,” Cell Signal 26:2186-2192 (2014), which is hereby incorporated by reference in its entirety). These include BAMBI as well as FST and GREM1, all acting in concert as negative feedback regulators of pro-gliogenic BMP signals (Brazil et al., “BMP Signaling: Agony and Antagony in the Family,” Trends Cell Biol 25:249-264 (2015); Onichtchouk et al., “Silencing of TGF-beta Signaling by the Pseudoreceptor BAMBI,” Nature 40:480-485 (1999), which are hereby incorporated by reference in their entirety) (FIG. 6A). On that basis, it was posited that SMAD4 knockdown in hGPCs, by inhibiting the early expression of BAMBI, FST and GREM1, might potentiate astrocytic differentiation from hGPCs. Furthermore, to the extent that the differentiation block in SCZ hGPCs was due to the SMAD4-mediated over-expression of endogenous BMP inhibitors, it was postulated that SMAD4 knock-down would therefore differentially potentiate astroglial differentiation by SCZ hGPCs. To test this possibility, doxycycline (DOX) induction of SMAD4 shRNAi was used to conditionally knock-down SMAD4 expression in both SCZ and CTR hGPCs, and then assessed their expression of BMP-regulated genes by qPCR (FIGS. 7A-7C). It was found that SMAD4 knockdown indeed repressed the expression of BMP signaling-dependent genes, including BAMBI, FST, and GREM1 (SCZ-LV-Scrambled vs SCZ-LV-SMAD4-shRNA; 4 different patient iPSC lines/group, 3 repeats/line; ddCt of BAMBI: 2.56±0.35, p<0.05; FST: 2.38±0.24, p<0.01; GREM1: 3.04±0.45, p<0.05; all comparisons by ANOVA with post hoc t tests) (FIG. 6B). Importantly, transient DOX-induced SMAD4 knockdown, in which shRNAi expression was limited to the progenitor stage, robustly promoted the astrocytic differentiation of the SCZ GPCs, overcoming their relative block in glial differentiation to effectively rescue astrocytic phenotype (FIGS. 6C-6D). In particular, SMAD4 knockdown (KD) in SCZ GPCs restored their efficiency of GFAP-defined astrocytic differentiation to that of CTR GPCs (SCZ-SMAD4-shRNA at the GPC stage: 56.8%±3.8%; CTR lines: 62.2%±4.0%; p>0.05, one-way ANOVA; means±SEs of 4 distinct patient lines/group, n≥3 replicates/line) (FIGS. 6C-6D). In contrast, continuous SMAD4 knock-down after astrocytic induction, as mediated via continuous DOX exposure (as outlined in FIG. 7B), caused a diminution of GFAP-defined astrocytes in both SCZ and CTR groups (FIGS. 6C-6D). Thus, maintenance of mature astrocytic phenotype appeared to require ongoing SMAD4 signaling, in SCZ and CTR astrocytes alike.

Together, these data indicate that aberrant BMP signaling in SCZ GPCs, by driving the excessive expression of inhibitors of BMP signaling, suppresses astrocytic differentiation, and that this differentiation defect can be rescued by SMAD4 knock-down. Nonetheless, once SCZ GPCs have progressed to astrocytic differentiation, SMAD4 expression is then required for maintenance of the astrocytic phenotype in CTR and SCZ astrocytes alike, consistent with its previously described function as the effector of BMP-mediated astrocytic maturation (Kohyama et al., “BMP-induced REST Regulates the Establishment and Maintenance of Astrocytic Identity,” J. Cell Biol. 189:159-170 (2010), which is hereby incorporated by reference in its entirety). These data indicate that pathological BMP-dependent signaling in SCZ GPs may delimit their astrocytic maturation, and suggest that this cellular pathology may arise in part from the SMAD4-dependent over-expression of endogenous inhibitors of pro-gliogenic BMP signaling by GPCs.

### Example 4—SCZ Astrocytes Exhibit Reduced Potassium Uptake

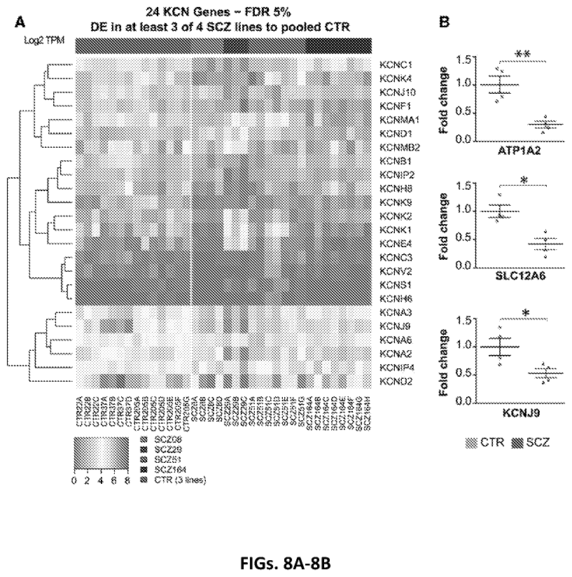

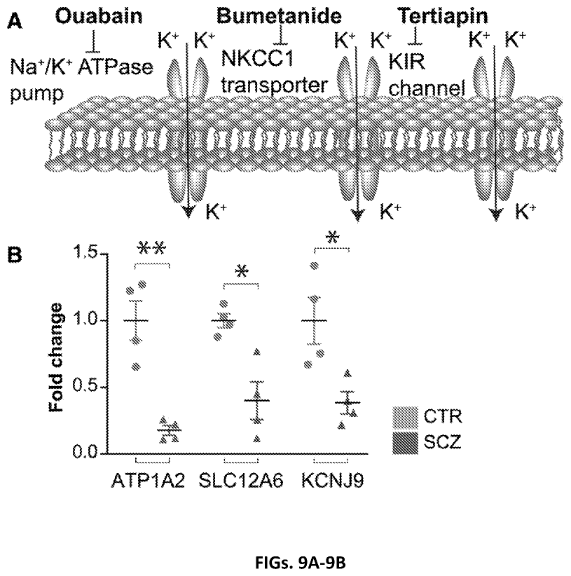

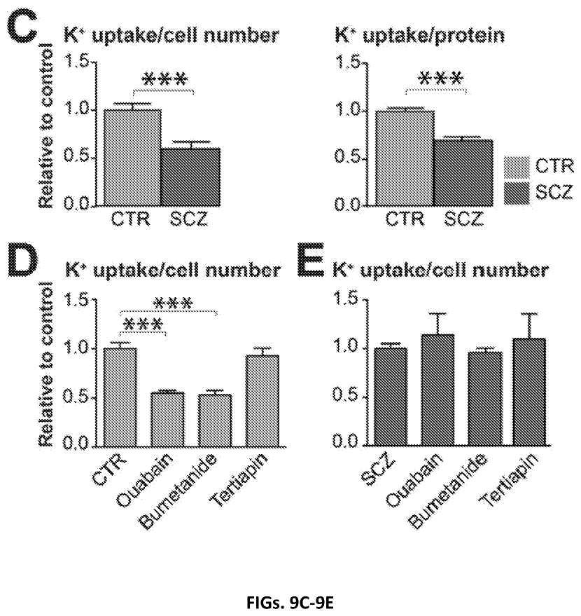

Together with the impaired astrocytic differentiation of SCZ GPCs, the RNA-seq data suggested that those astrocytes that do successfully differentiate might nonetheless be functionally impaired. In particular, the RNA-seq revealed the downregulated transcription in SCZ GPCs of a broad set of potassium channel (KCN)-encoding genes, including the Na⁺—K⁺ ATPase, Na⁺-K⁺/2Cl⁻ cotransporter (NKCC), and Kir-family inwardly rectifying potassium channels (FIG. 8A) (Windrem et al., “Human iPSC Glial Mouse Chimeras Reveal Glial Contributions to Schizophrenia,” Cell Stem Cell 21:195-208.e6 (2017), which is hereby incorporated by reference in its entirety), all of which play important roles in potassium uptake by astrocytes (Larsen et al., “Contributions of the Na(+)/K(+)-ATPase, NKCC1, and Kir4.1 to Hippocampal K(+) Clearance and Volume Responses,” Glia 62:608-622 (2014); Macaulay & Zeuthen, “Glial K(+) Clearance and Cell Swelling: Key Roles for Cotransporters and Pumps,” Neurochem. Res. 37:2299-2309 (2012), which are hereby incorporated by reference in their entirety) (FIG. 9A). Among these dysregulated KCN genes, ATP1A2, SLC12A6, and KCNJ9, which respectively encode the Na⁺/K⁺-ATPase pump, NKCC1 Na⁺/K⁺/2Cl⁻ cotransporter, and the Kir3.3 voltage gated K⁺ channel (Bottger et al., “Glutamate—System Defects Behind Psychiatric Manifestations in a Familial Hemiplegic Migraine Type 2 Disease-Mutation Mouse Model,” Sci. Rep. 6:22047 (2016); Gamba & Friedman, “Thick Ascending Limb: The Na(+):K (+):2Cl (−) Cotransporter, NKCC2, and the Calcium-Sensing Receptor, CaSR,” Pflugers Arch. 458: 61-76 (2009); Lesage et al., “Molecular Properties of Neuronal G-Protein-Activated Inwardly Rectifying K+ Channels,” J. Biol. Chem. 270:28660-28667 (1995), which are hereby incorporated by reference in their entirety), were consistently and substantially down-regulated in all 4 SCZ lines assessed, compared to the 4 control lines. These findings suggested a broad-based impairment of K+ uptake by SCZ glia.

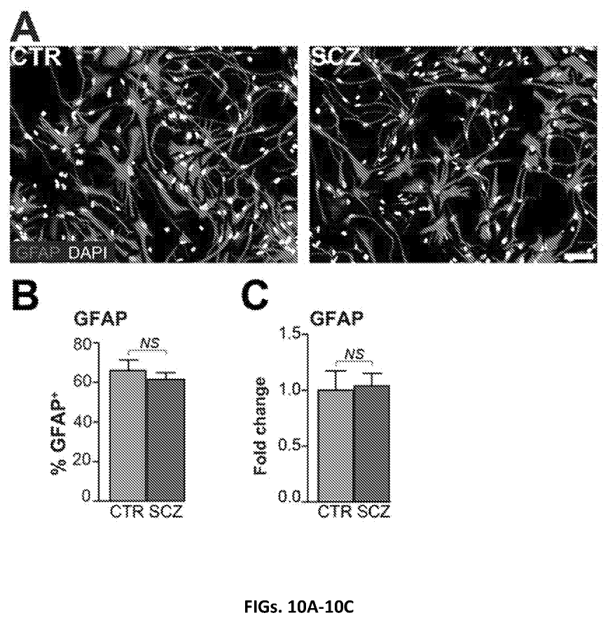

On the basis of these genomic data, whether K⁺ uptake was actually impaired in SCZ astrocytes was assessed. To address this hypothesis, qPCR was used to confirm whether these K+ channel-associated genes were dysregulated in SCZ glia. They were indeed significantly down regulated, thus validating the RNA-seq analysis (FIG. 8B and FIG. 9B). Next, functional K⁺ uptake was assessed directly, in cultured SCZ- and CTR-derived astrocytes. To obtain mature SCZ and CTR astrocyte cultures, CD44− sorted glial progenitors were cultured in base media supplemented with 10% fetal bovine serum (FBS) and 20 ng/ml BMP4 for 4 weeks, so as to potentiate the differentiation of mature, glial fibrillary acidic protein (GFAP)-expressing, fiber-bearing astrocytes (FIGS. 10A-10C). Under these highly astrogliogenic conditions, and using cells already sorted for the early astrocytic marker CD44, astrocytic maturation was achieved by both SCZ- as well as CTR derived progenitor cells (FIGS. 10A-10C). Astrocytes from 4 different SCZ and 4 different CTR lines were then incubated with 86Rb, a surrogate monovalent cation for K⁺ uptake (Larsen et al., “Contributions of the Na(+)/K(+)-ATPase, NKCC1, and Kir4.1 to Hippocampal K(+) Clearance and Volume Responses,” Glia 62:608-622 (2014), which is hereby incorporated by reference in its entirety), and rubidium uptake measured as a function of both cell number and total protein. The K+ uptake in SCZ glia (4 SCZ cell lines, 5 repeats/each line) was sharply decreased relative to CTR glia (4 CTR cell lines, 5 repeats/each line), normalized by both cell number and total protein (FIG. 9C; P<0.001 by two tailed t-test).

Since genes encoding different potassium Na⁺/K⁺-ATPase pumps, and inwardly rectifying channels were dysregulated in SCZ glia, the drugs ouabain, bumetanide, and tertiapin were used to respectively block these three potassium uptake mechanisms. The actions of these drugs on astrocytes had not been previously assessed, so different concentrations of each were first tested to determine optimal dose ranges for modulating human astroglial K⁺ uptake. Ouabain and bumetanide, respectively, targeting the Na⁺/K⁺-ATPase pump and NKCC1-encoded Na⁺/K⁺/2Cl⁻ cotransporter, significantly inhibited K⁺ uptake in CTR glia, while tertiapin, which targets Kir channels, did not (FIGS. 9D-9E, left graphs). In marked contrast, neither ouabain nor bumetanide affected K⁺ uptake by SCZ astrocytes (FIGS. 9D-9E, right graphs). This suggests that the functional decrement in K⁺ uptake by SCZ-derived astrocytes may be primarily due to down-regulated Na⁺/K⁺-ATPase and Na⁺/K⁺/2Cl⁻ cotransporter function, rendering these cells refractory to ouabain and bumetanide treatment.

### Discussion of Examples 1-4

These data indicate that astrocytic differentiation is impaired in GPCs derived from childhood-onset schizophrenics, and that this maturational defect may be rescued by the suppression of either BMP signaling via SMAD4 knock-down. Importantly, astrocytic depletion has been recently noted in both cortical and subcortical regions of patients with schizophrenia, and this might be especially prominent in the white matter, (Rajkowska et al., “Layer-specific Reductions in GFAP-reactive Astroglia in the Dorsolateral Prefrontal Cortex in Schizophrenia,” Schizophr Res 57:127-138 (2002); Steffek et al., “Cortical Expression of Glial Fibrillary Acidic Protein and Glutamine Synthetase is Decreased in Schizophrenia,” Schizophr Res 103:71-82 (2008); Williams et al., “Astrocyte Decrease in the Subgenual Cingulate and Callosal Genu in Schizophrenia,” Eur Arch Psychiatry Clin Neurosci 263:41-52 (2013), which are hereby incorporated by reference in their entirety). Astrocytes play key contributions to neural circuit formation and stability (Christopherson et al., “Thrombospondins are Astrocyte-secreted Proteins that Promote CNS Synaptogenesis,” Cell 120:421-433 (2005); Clarke and Barres, “Emerging Roles of Astrocytes in Neural Circuit Development,” Nature Reviews Neuroscience 14:311-321 (2013), which are hereby incorporated by reference in their entirety). Thus, any such developmental defect of astrocytic differentiation in SCZ GPCs might lead to profound defects in the initial formation or stability of neural circuits, a defect that is one of the hallmarks of schizophrenia (Penzes et al., “Dendritic Spine pathology in Neuropsychiatric Disorders,” Nat Neurosci 14:285-293 (2011), which is hereby incorporated by reference in its entirety). In this regard, the RNA-seq data suggested upregulated TGFBR and BMP signaling in SCZ GPCs, which was associated with the activation of downstream BMP-regulated genes that included BAMBI, a competitive inhibitor of pro-gliogenic BMP signaling (Onichtchouk et al., “Silencing of TGF-beta Signaling by the Pseudoreceptor BAMBI,” Nature 40:480-485 (1999), which is hereby incorporated by reference in its entirety). It has been previously noted that high-expression of BAMBI in adult human GPCs significantly inhibits their astrocytic differentiation as induced by BMP4 (Sim et al., “Complementary Patterns of Gene Expression by Human Oligodendrocyte Progenitors and Their Environment Predict Determinants of Progenitor Maintenance and Differentiation,” Ann Neurol 59:763-779 (2006), which is hereby incorporated by reference in its entirety), suggesting that the pathological elevation of BMP signaling-induced BAMBI expression in SCZ hGPCs, relative to normal control hGPCs, might be sufficient to suppress their differentiation as mature astrocytes. Besides BAMBI, several other inhibitors of TGFβ/BMP signaling, including FST and GREM1 (Brazil et al., “BMP Signalling: Agony and Antagony in the Family,” Trends Cell Biol 25:249-264 (2015), which is hereby incorporated by reference in its entirety), were also upregulated by SCZ GPCs; these may have permitted SCZ hGPCs to avoid astrocytic fate even after BAMBI knockdown.

Of note, the activation of canonical TGFβ signaling is dependent upon either SMAD2/3 activation via the TGFβ pathway, or SMAD1/5/8 via BMP receptor-dependent signals; each of these effectors needs to combine with SMAD4 for nuclear translocation prior to the activation of their downstream genetic targets (Hata and Chen, “TGF-beta Signaling from Receptors to Smads,” Cold Spring Harb Perspect Biol 8 (2016); Herhaus and Sapkota, “The Emerging Roles of Deubiquitylating Enzymes (DUBS) in the TGFbeta and BMP Pathways,” Cell Signal 26:2186-2192 (2014), which are hereby incorporated by reference in their entirety). Accordingly, it was found that SMAD4 knockdown efficiently suppressed BMP signaling-induced expression of endogenous BMP inhibitors, and by so doing robustly promoted the astrocytic differentiation of otherwise differentiation-resistant SCZ GPCs. Importantly, this differentiative response of hGPCs to SMAD4 inhibition was only noted at the hGPC stage, and only in SCZ hGPCs; control patient-derived hGPCs showed no such potentiated differentiation in response to SMAD4 suppression. Thus, the modulation of SMAD4 might represent an appropriate strategy towards relieving the glial differentiation defect in schizophrenia.

Glial maturation is precisely regulated in human brain development (Goldman and Kuypers, “How to Make an Oligodendrocyte,” Development 142:3983-3995 (2015); Molofsky et al., “Astrocytes and Disease: a Neurodevelopmental Perspective,” Genes and Development 26:891-907 (2012), which are hereby incorporated by reference in their entirety). Astrocytes have a multitude of roles in the CNS, including energy support to both neurons and oligodendrocytes, potassium buffering, neurotransmitter recycling, and synapse formation and maturation; as such, astrocytes play critical roles in neural circuit formation and maintenance (Blanco-Suarez et al., “Role of Astrocyte-synapse Interactions in CNS Disorders,” J. Physiol. 595:1903-1916 (2017); Clarke and Barres, “Emerging Roles of Astrocytes in Neural Circuit Development,” Nature Reviews Neuroscience 14:311-321 (2013); Verkhratsky et al., “Why are Astrocytes Important? Neurochemical Research 40:389-401 (2015), which are hereby incorporated by reference in their entirety). Astrocytes also contribute to the lymphatic system, through the regulation of cerebral spinal fluid flow through the brain interstitium (Xie et al., “Sleep Drives Metabolic Clearance from the Adult Brain,” Science 342:373-377 (2013), which is hereby incorporated by reference in its entirety). Thus, the delayed differentiation of SCZ astrocytes may have significant effects on neural network formation, organization and mature function alike.

It was found that a number of potassium transporters were down-regulated in SCZ glia. Interestingly, prior genome wide association studies (GWAS) have identified an association of potassium pump, transport and channel genes with schizophrenia. For instance, the chromosome 1q21-q22 locus, containing KCNN3, has a significant linkage to familial schizophrenia (Brzustowicz et al., “Location of a Major Susceptibility Locus for Familial Schizophrenia on Chromosome 1q21-q22,” Science 288:678-682 (2000), which is hereby incorporated by reference in its entirety). KCNN3 is widely expressed in the human brain, and selectively regulates neuronal excitability and neurotransmitter release in monoaminergic neurons (O'Donovan and Owen, “Candidate-gene Association Studies of Schizophrenia,” Am. J. Hum. Genet. 65:587-592 (1999), which is hereby incorporated by reference in its entirety). In addition to KCNN3, a number of other potassium channel genes have been associated with schizophrenia, including KCNQ2 and KCNAB1 (Lee et al., “Pathway Analysis of Genome-wide Association Study in Schizophrenia,” Gene 525:107-115 (2013), which is hereby incorporated by reference in its entirety). More recently, a novel de novo mutation in ATP1A3, a subunit of the sodium-potassium pump, has been specifically associated with childhood-onset schizophrenia (Smedemark-Margulies et al., “A Novel De Novo Mutation in ATP1A3 and Childhood-Onset Schizophrenia,” Cold Spring Harb Mol Case Stud 2, a001008 (2016), which is hereby incorporated by reference in its entirety).

The down-regulation or dysfunction of these potassium transporters in GPCs and their derived astrocytes may contribute significantly to disease phenotype in schizophrenia. Potassium channel, pump and transport genes are widely expressed in both GPCs (Coppi et al., “UDP-glucose Enhances Outward K(+) Currents Necessary for Cell Differentiation and Stimulates Cell Migration by Activating the GPR17 Receptor in Oligodendrocyte Precursors,” Glia 61:1155-1171 (2013); Maldonado et al., “Oligodendrocyte Precursor Cells are Accurate Sensors of Local K⁺ in Mature Gray Matter,” J. Neurosci. 33:2432-2442 (2013), which are hereby incorporated by reference in their entirety) and astrocytes (Larsen et al., “Contributions of the Na(+)/K(+)-ATPase, NKCC1, and Kir4.1 to Hippocampal K(+) Clearance and Volume Responses,” Glia 62:608-622 (2014); Zhang and Barres, “Astrocyte Heterogeneity: an Underappreciated Topic in Neurobiology,” Current Opinion in Neurobiology 20:588-594 (2010), which are hereby incorporated by reference in their entirety), in which they regulate not only proliferation, migration, and differentiation, but also the relationship of glia to neurons (Coppi et al., “UDP-glucose Enhances Outward K(+) Currents Necessary for Cell Differentiation and Stimulates Cell Migration by Activating the GPR17 Receptor in Oligodendrocyte Precursors,” Glia 61:1155-1171 (2013); Maldonado et al., “Oligodendrocyte Precursor Cells are Accurate Sensors of Local K⁺ in Mature Gray Matter,” J. Neurosci. 33:2432-2442 (2013), which are hereby incorporated by reference in their entirety). In regards to the latter, astrocytes also regulate synaptic K⁺ uptake through all three major K⁺ transport mechanisms, including the Na+/K+-ATPase, the NKCC1 cotransporter, and inwardly rectifying Kir channels (Larsen et al., “Contributions of the Na(+)/K(+)-ATPase, NKCC1, and Kir4.1 to Hippocampal K(+) Clearance and Volume Responses,” Glia 62:608-622 (2014); Zhang and Barres, “Astrocyte Heterogeneity: an Underappreciated Topic in Neurobiology,” Current Opinion in Neurobiology 20:588-594 (2010), which are hereby incorporated by reference in their entirety), thereby establishing neuronal firing thresholds over broad regional domains.

Accordingly, dysregulated K+ transport and potassium channel gene expression have been associated with a broad variety of neurological and psychiatric diseases. Several Kir genes, including Kir4.1, are involved in astrocytic potassium buffering and glutamate uptake, and deletion of these genes has been noted in both Huntington's disease and multiple sclerosis (Seifert et al., “Astrocyte Dysfunction in Neurological Disorders: a Molecular Perspective,” Nat Rev Neurosci 7:194-206 (2006); Tong et al., “Astrocyte Kir4.1 Ion Channel Deficits Contribute to Neuronal Dysfunction in Huntington's Disease Model Mice,” Nature Neuroscience 17:694-703 (2014), which are hereby incorporated by reference in their entirety). In addition, mutation of astrocytic ATP1A2, the a2-isoform of the sodium-potassium pump, may be causally associated with familial hemiplegic migraine (Bottger et al., “Glutamate-system Defects Behind Psychiatric Manifestations in a Familial Hemiplegic Migraine Type 2 Disease-mutation Mouse Model,” Sci Rep 6:22047 (2016); Swarts et al., “Familial Hemiplegic Migraine Mutations Affect Na,K-ATPase Domain Interactions,” Biochim Biophys Acta 1832:2173-2179 (2013), which are hereby incorporated by reference in their entirety). In all of these examples, glial K⁺ uptake is impaired, just as in SCZ glia, and all are associated with elements of phenotypic hyperexcitability. Indeed, elevated extracellular K⁺ has been shown to alter the neuronal excitability and neural circuit stability in a mouse model of schizophrenia (Crabtree et al., “Alteration of Neuronal Excitability and Short-term Synaptic Plasticity in the Prefrontal Cortex of a Mouse Model of Mental Illness,” J. Neurosci. (2017), which is hereby incorporated by reference in its entirety). Thus, the decreased K⁺ uptake of SCZ glia may be a significant contributor to schizophrenia pathogenesis, especially in regards to those schizophrenic phenotypes associated with hyperexcitability and seizure disorders, which would be potentiated in the setting of disrupted potassium homeostasis.

Thus, these data reveal the defective differentiation into astrocytes by SCZ GPCs, the potential reversibility of that defect by SMAD4 knockdown, and the defective uptake of K+ by SCZ glia. The resultant deficiencies in synaptic potassium homeostasis may be expected to significantly lower neuronal firing thresholds while accentuating network desynchronization (Benraiss et al., “Human Glia Can Both Induce and Rescue Aspects of Disease Phenotype in Huntington Disease,” Nature Communications 7:11758 (2016), which is hereby incorporated by reference in its entirety). As such, one might expect that positive modulators of glial K⁺ uptake may have real value in the treatment of schizophrenia (Calcaterra et al., “Schizophrenia-associated hERG Channel Kv11.1-3.1 Exhibits a Unique Trafficking Deficit that is Rescued Through Proteasome Inhibition for High Throughput Screening,” Sci Rep 6:19976 (2016); He et al., “Current Pharmacogenomic Studies on hERG Potassium Channels,” Trends Mol Med 19:227-238 (2013); Rahmanzadeh et al., “Lack of the Effect of Bumetanide, a Selective NKCC1 Inhibitor, in Patients with Schizophrenia: A Double-blind Randomized Trial,” Psychiatry Clin Neurosci 71:72-73 (2017), which are hereby incorporated by reference in their entirety). Together, these findings identify a causal contribution of astrocytic pathology to the neuronal dysfunction of SCZ, and in so doing suggest a set of tractable molecular targets for its treatment.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of restoring K+ uptake by glial cells having impaired K⁺ channel function, said method comprising
administering, to the glial cells having impaired K⁺ channel function, a SMAD4 inhibitor under conditions effective to restore K⁺ uptake by said glial cells.

2. The method of claim 1, wherein the glial cells are glial progenitor cells.

3. The method of claim 2, wherein said administering is carried out under conditions effective to restore glial progenitor cell astrocyte differentiation.

4. The method of claim 1, wherein the glial cells are astrocytes.

5. The method of claim 1, wherein the SMAD4 inhibitor is an inhibitory nucleic acid molecule selected from the group consisting of a SMAD4 antisense oligonucleotide, a SMAD4 shRNA, and a SMAD4 siRNA.

6. The method of claim 1, wherein the SMAD4 inhibitor is a small molecule selected from the group consisting of 5-Fluorouracil, valproic acid, vorinostat, and PR-629.

7. The method of claim 1, wherein the glial cells having impaired K+ channel activity are glial cells of a subject having a neuropsychiatric disorder.

8. The method of claim 8, wherein the neuropsychiatric disorder is schizophrenia.

9. A method of restoring K⁺ uptake by glial cells in a subject, said method comprising:
selecting a subject having impaired glial cell K⁺ uptake, and
administering, to the selected subject, a SMAD4 inhibitor under
conditions effective to restore K⁺ uptake by said glial cells.

10. The method of claim 9, wherein the glial cells are glial progenitor cells.

11. The method of claim 10, wherein said administering is carried out under conditions effective to restore glial progenitor cell astrocyte differentiation in the subject.

12. The method of claim 9, wherein the glial cells are astrocytes.

13. The method of claim 12, wherein said administering is carried out under conditions effective to restore astrocyte K⁺ homeostasis.

14. The method of claim 9, wherein the SMAD4 inhibitor is an inhibitory nucleic acid molecule selected from the group consisting of a SMAD4 antisense oligonucleotide, a SMAD4 shRNA, and a SMAD4 siRNA.

15. The method of claim 9, wherein the SMAD4 inhibitor is a small molecule selected from the group consisting of 5-Fluorouracil, valproic acid, vorinostat, and PR-629.

16. The method of claim 9, wherein the SMAD4 inhibitor is packaged in a nanoparticle delivery vehicle.

17. The method of claim 15, wherein the delivery vehicle comprises a glial cell targeting moiety.

18. The method of claim 9, wherein the selected subject has or is at risk of having a neuropsychiatric disorder.

19. The method of claim 18, wherein the neuropsychiatric disorder is selected from the group consisting of schizophrenia, autism spectrum disorder, and bipolar disorder.

20. The method of claim 19, wherein the neuropsychiatric disorder is schizophrenia.

21. The method of claim 9, wherein said administering is carried out under conditions effective to decrease neuronal excitability in said subject.

22. The method of claim 9, wherein said administering is carried out under conditions effective to decreases seizure incidence in said subject.

23. The method of claim 9, wherein said is carried out under conditions effective to administering improves disordered cognition in said subject.

24. The method of claim 9, wherein said administering is carried out using intracerebral delivery, intrathecal delivery, intranasal delivery, or via direct infusion into brain ventricles.

25. The method of claim 9, wherein the subject is human.

26. A method of treating or inhibiting the onset of a neuropsychiatric disorder in a subject, said method comprising:
selecting a subject having or at risk of having a neuropsychiatric disorder, and
administering, to the selected subject, a SMAD4 inhibitor under conditions effective to treat or inhibit the onset of the neuropsychiatric disorder in the subject.

27.-40. (canceled)
